(19) United States
(12) Patent Application Publication
(10) Pub. No.: US 2025/0082376 A1
(43) Pub. Date: Mar. 13, 2025

(54) EXPANDABLE SYSTEM FOR REPAIR OF BONE FRACTURES

(71) Applicant: MiRus LLC, Marietta, GA (US)

(72) Inventor: Jay Yadav, Atlanta, GA (US)

(21) Appl. No.: 18/540,271

(22) Filed: Dec. 14, 2023

Related U.S. Application Data
(60) Provisional application No. 63/536,948, filed on Sep. 7, 2023.

(51) Int. Cl. A61B 17/72; A61L 31/02; A61L 31/08; A61L 31/14

(52) U.S. Cl. CPC A61B 17/7275; A61L 31/022; A61L 31/088; A61L 31/148; A61B 2017/00004; A61B 2017/564

(57) ABSTRACT

A system for percutaneous fixation and stabilization of a fracture with a spanning, expandable structural frame placed in the intramedullary canal of the bone comprising an expandable medical device.

### REFERENCED APPLICATIONS

The present application claims priority to U.S. Provisional Application Ser. No. 63/536,948 filed Sep. 7, 2023, which is incorporated herein by reference.

### FIELD OF DISCLOSURE

The present disclosure relates generally to the fixation, repair and stabilization of bone fractures, particularly to an expandable medical device that can be partially or fully inserted into a fractured bone to facilitate in the fixation, repair and stabilization of the fractured bone, more particularly to an expandable medical device that is partially or fully formed of a rhenium containing metal alloy that can be partially or fully inserted into a fractured bone to facilitate in the fixation, repair and stabilization of the fractured bone, and still even more particularly to an expandable medical device that is partially or fully formed of a rhenium containing metal alloy that can be partially or fully inserted into a fractured bone to facilitate in the fixation, repair and stabilization of the fractured bone, and wherein the metal alloy used to partially or fully form the expandable medical device has a sufficient quantity of rhenium such that the ductility and tensile strength of the metal alloy is improved.

### BACKGROUND

The common methods of treating bone fractures ranges from the setting of the bone fracture to constraining the motion in the area of the bone fracture via a cast or wrap. Commonly, pins, screws, rods and cement are used to repair fracture bone. In some of these bone fracture treatments, the fractured bone is not properly stabilized, thereby resulting in potential misalignment of the fractured bone. FIGS. 1-3 illustrate common systems and methods for the fixation of bone fractures include external immobilization of the fracture with casts, splinting devices or external fixation frames (See FIG. 1), internal fixation with plates and screws (See FIG. 2), or indirect fixation of the fracture by insertion of an intramedullary device (See FIG. 3).

As described and illustrated in FIGS. 1-3, these prior art bone fixation devices have several disadvantages. For instance, casting devices as illustrated in FIG. 1 can result in a) prolonged immobilization of the fractures region, thus adding discomfort and inconvenience to the patient during the healing of the fracture bone, b) can result in muscle atrophy due to prolonged immobilization, thus requiring extended recover periods and potential physical therapy, c) can increase incidence of thrombosis, and d) can result in disuse syndrome.

Some bone fixation devices currently use compression plates and screw devices to apply a compression force across the fracture site (See FIG. 2). Such fixation devices sometimes require a large surgical incision over the bone at the fracture site. The installing of the plates and screws usually a) requires the disturbance of the soft tissues overlying the fracture site, b) causes a disturbance of the fracture hematoma, c) is a highly invasive procedure, d) can result in s risk of vascular lesions, e) can require the use of extracortical hardware for later removal of the fixation device, and/or f) can result in the stripping of the periosteum of bone which can result in compromises to the blood supply of the fracture bone fragments.

As illustrated in FIG. 3, another system for treating a bone fracture site includes intramedullary nailing. In such a procedure, one or more nails are inserted into the intramedullary canal of a fractured bone, usually through an incision located at either end of the bone. Intramedullary nailing can be advantages over external casting and other methods of fracture stabilization. However, there are several disadvantages associated with intramedullary nailing, namely a) the procedure is highly invasive, b) requires a large incision into the bone (e.g., about 15 mm), c) can result in cortical reaming, d) results in substantial or full bone marrow displacement, c) can result in a risk of pulmonary embolism, f) the reaming of the intramedullary canal and the placement of a nail without reaming can compromise the intramedullary blood supply to the bone fracture, g) the shape of nail can be non-confirming to the shape of the intramedullary canal thereby resulting in improper or difficult placement of the nail in the canal, and/or h) hardware migration can result during the repair of the bone fracture, thereby resulting in slower recovery times and improper fracture repair. Several prior art intramedullary nails are illustrated in U.S. Pat. Nos. 6,783,530; 6,551,321; 6,224,600; US 2009/0018542; US 2008/0255560; and US 2002/0032444, all of which are incorporated by reference herein.

In view of the current prior art, there is a need in the art for a less invasive and more effective method of stabilizing a bone fracture site with minimal disruption of the fracture biology, reduced trauma to the intramedullary canal, better biomechanical properties, and smaller incisions.

### SUMMARY OF THE DISCLOSURE

The present disclosure relates generally to the fixation, repair and stabilization of bone fractures, particularly to an expandable medical device that can be partially or fully inserted into a fractured bone to facilitate in the fixation, repair and stabilization of the fractured bone, more particularly to an expandable medical device that is partially or fully formed of a rhenium containing metal alloy that can be partially or fully inserted into a fractured bone to facilitate in the fixation, repair and stabilization of the fractured bone, and still even more particularly to an expandable medical device that is partially or fully formed of a rhenium containing metal alloy that can be partially or fully inserted into a fractured bone to facilitate in the fixation, repair and stabilization of the fractured bone, and wherein the metal alloy used to partially or fully form the expandable medical device has a sufficient quantity of rhenium such that the ductility and tensile strength of the metal alloy is improved. In one non-limiting embodiment, the expandable medical device is generally configured to be partially or fully inserted into a canal of a fractured bone, has a sufficient length to span the fracture site of the bone fracture, and is expandable in the canal so as to conform to the internal surface of the bone canal. In another non-limiting embodiment, a guidewire is optionally provided for use with the expandable medical device. The guidewire can be used to facilitate in the insertion of the expandable medical device into the bone canal of the fractured bone. The guidewire can have a size and shape suitable for insertion into the bone canal, a length sufficient to span the fracture site of the fractured bone, and sufficient flexibility and support to guide the expandable medical device partially or fully through the bone canal of the fractured bone. In another non-limiting embodiment, a sheath is optionally provided for use with the expandable medical device. The sheath can be used to facilitate in the insertion of the expandable medical device into the bone canal of the fractured bone. The sheath can have a size and shape suitable for insertion into the bone canal, a length sufficient to span the fracture site of the fractured bone, and/or an elongate longitudinal cavity that is sized and shaped to receive the expandable medical device partially or fully through the cavity. In another non-limiting embodiment, a hardenable surgical fluid can optionally be used with the expandable medical device. The hardenable surgical fluid, when used, provides additional support across the fracture site when used with the expandable medical device.

In another and/or alternative non-limiting aspect of the disclosure, the expandable medical device is optionally partially or fully formed of a) a refractory metal alloy and/or b) a metal alloy that includes at least 15 atomic weight percent (awt. %) or atomic percent (awt %) rhenium so as to create a “rhenium effect” in the metal alloy. As used herein, atomic weight percent (awt. %) or atomic percent (awt %) are used interchangeably. As defined herein, the weight percentage (wt. %) of an element is the weight of that element measured in the sample divided by the weight of all elements in the sample multiplied by 100. The atomic percentage or atomic weight percent (awt %) is the number of atoms of that element, at that weight percentage, divided by the total number of atoms in the sample multiplied by 100. The use of the terms weight percentage (wt. %) and atomic percentage or atomic weight percentage (awt. %) are two ways of referring to metallic alloy and its constituents. It has been found that for several metal alloys the inclusion of at least 15 awt. % rhenium results in the ductility and/or tensile strength of the metal alloy to improve as compared to a metal alloy is that absent rhenium. Such improvement in ductility and/or tensile strength due to the inclusion of at least 15 awt. % rhenium in the metal alloy is referred to as the “rhenium effect.” As defined herein, a “rhenium effect” is a) an increase of at least 10% in ductility of the metal alloy caused by the addition of rhenium to the metal alloy, and/or b) an increase of at least 10% in tensile strength of the metal alloy caused by the addition of rhenium to the metal alloy. As defined herein, a refractory metal alloy is a metal alloy that includes at least 20 wt. % of one or more of molybdenum, rhenium, niobium, tantalum or tungsten. Non-limiting refractory metal alloys include MoRe alloy, ReW alloy, MoReCr alloy, MoReTa alloy, MoReTi alloy, WCu alloy, ReCr, molybdenum alloy, rhenium alloy, tungsten alloy, tantalum alloy, niobium alloy, etc. In one non-limiting arrangement, 50-100 wt. % (and all values and ranges therebetween) of the expandable medical device is formed of a refractory metal alloy or a metal alloy that includes at least 15 awt. % rhenium. In another non-limiting arrangement, the metal alloy that is used to partially or fully form the expandable medical device includes at least 30 wt. % (e.g., 30-99 wt. % and all values and ranges therebetween) of one or more of molybdenum, rhenium, niobium, tantalum or tungsten. In another non-limiting embodiment, the refractory metal alloy or the metal alloy that includes at least 15 awt. % rhenium can be used to 1) increase the radiopacity of the expandable medical device, 2) increase the radial strength of the expandable medical device, 3) increase the yield strength and/or ultimate tensile strength of the expandable medical device, 4) improve the stress-strain properties of the expandable medical device, 5) improve the crimping and/or expansion properties of the expandable medical device, 6) improve the bendability and/or flexibility of the expandable medical device, 7) improve the strength and/or durability of the expandable medical device, 8) increase the hardness of the expandable medical device, 9) improve the biostability and/or biocompatibility properties of the expandable medical device, 10) increase fatigue resistance of the expandable medical device, 11) resist cracking in the expandable medical device, 12) resist propagation of cracks in the expandable medical device, 13) enable smaller, thinner, and/or lighter weight expandable medical device to be made, 14) facilitate in the reduction of the outer diameter of a crimped expandable medical device, 15) improve the conformity of the expandable medical device to the shape of the treatment area when the expandable medical device is expanded in the treatment area, 16) reduce the amount of recoil of the expandable medical device after the expandable medical device is expanded in the treatment area, 17) reduce adverse tissue reactions with the expandable medical device, 18) reduce metal ion release from the expandable medical device after implantation of the expandable medical device, 19) reduce corrosion of the expandable medical device after implantation of the expandable medical device, 20) reduce allergic reaction with the expandable medical device after implantation of the expandable medical device (e.g., reduce nickel content of metal alloy, etc.), 21) improve hydrophilicity of the expandable medical device, 22) reduce magnetic susceptibility of the expandable medical device, and/or 23) reduce toxicity of the expandable medical device after implantation of the expandable medical device.

In another and/or alternative non-limiting aspect of the disclosure, the expandable medical device is optionally partially or fully formed of standard stainless steel, standard CoCr alloys, standard TiAlV alloys, standard aluminum alloys, standard nickel alloys, standard titanium alloys, standard tungsten alloys, standard molybdenum alloys, standard copper alloys, standard MP35N alloys, standard beryllium-copper alloys that have been modified to include at least 15 awt. % rhenium so as to result in improved ductility and/or tensile strength as compared to the same metal alloy that is absent rhenium. As defined herein, a standard stainless-steel alloy (SS alloy) includes 10-28 wt. % (weight percent) chromium, 0-35 wt. % nickel, 0-4 wt. % molybdenum, 0-2 wt. % manganese, 0-0.75 wt. % silicon, 0-0.3 wt. % carbon, 0-5 wt. % titanium, 0-10 wt. % niobium, 0-5 wt. % copper, 0-4 wt. % aluminum, 0-10 wt. % tantalum, 0-1 wt. % Se, 0-2 wt. % vanadium, 0-2 wt. % tungsten, and at least 50 wt. % iron. A standard 316L alloy that falls within a standard stainless-steel alloy includes 17-19 wt. % chromium, 13-15 wt. % nickel, 2-4 wt. % molybdenum, 2 wt. % max manganese, 0.75 wt. % max silicon, 0.03 wt. % max carbon, balance iron. As defined herein, a standard cobalt-chromium alloy (CoCr alloy) includes 15-32 wt. % chromium, 1-38 wt. % nickel, 2-18 wt. % molybdenum, 0-18 wt. % iron, 0-1 wt. % titanium, 0-0.15 wt. % manganese, 0-0.15 wt. % silver, 0-0.25 wt. % carbon, 0-16 wt. % tungsten, 0-2 wt. % silicon, 0-2 wt. % aluminum, 0-1 wt. % iron, 30-68 wt. % cobalt, 0-0.1 wt. % boron, 0-0.15 wt. % silver, and 0-2 wt. % titanium. As a standard MP35N alloy that falls within a standard CoCr alloy includes 18-22 wt. % chromium, 32-38 wt. % nickel, 8-12 wt. % molybdenum, 0-2 wt. % iron, 0-0.5 wt. % silicon, 0-0.5 wt. % manganese, 0-0.2 wt. % carbon, 0-2 wt. % titanium, 0-0.1 wt. %, 0-0.1 wt. % boron, 0-0.15 wt. % silver, and balance cobalt. As defined herein, a standard Phynox and standard Elgiloy alloy that falls within a standard CoCr alloy includes 38-42 wt. % cobalt, 18-22 wt. % chromium, 14-18 wt. % iron, 13-17 wt. % nickel, 6-8 wt. % molybdenum. As defined herein, a standard L605 alloy that falls within a standard CoCr alloy includes 18-22 wt. % chromium, 14-16 wt. % tungsten, 9-11 wt. % nickel, balance cobalt. As defined herein, a standard titanium-aluminum-vanadium alloy (TiAlV alloy) includes 5.5-6.75 wt. % aluminum, 3.5-4.5 wt. % vanadium, 85-93 wt. % titanium, 0-0.4 wt. % iron, 0-0.2 wt. % carbon. A standard Ti-6Al-4V alloy that falls with a standard TiAlV alloy includes incudes 3.5-4.5 wt. % vanadium, 5.5-6.75 wt. % aluminum, 0.3 wt. % max iron, 0.08 wt. % max carbon, 0.05 wt. % max yttrium, balance titanium. As defined herein, a standard aluminum alloy includes 80-99 wt. % aluminum, 0-12 wt. % silicon, 0-5 wt. % magnesium, 0-1 wt. % manganese, 0-0.5 wt. % scandium, 0-0.5 wt. % beryllium, 0-0.5 wt. % yttrium, 0-0.5 wt. % cerium, 0-0.5 wt. % chromium, 0-3 wt. % iron, 0-0.5, 0-9 wt. % zinc, 0-0.5 wt. % titanium, 0-3 wt. % lithium, 0-0.5 wt. % silver, 0-0.5 wt. % calcium, 0-0.5 wt. % zirconium, 0-1 wt. % lead, 0-0.5 wt. % cadmium, 0-0.05 wt. % bismuth, 0-1 wt. % nickel, 0-0.2 wt. % vanadium, 0-0.1 wt. % gallium, and 0-7 wt. % copper. As defined herein, a standard nickel alloy includes 30-98 wt. % nickel, 5-25 wt. % chromium, 0-65 wt. % iron, 0-30 wt. % molybdenum, 0-32 wt. % copper, 0-32 wt. % cobalt, 2-2 wt. % aluminum, 0-6 wt. % tantalum, 0-15 wt. % tungsten, 0-5 wt. % titanium, 0-6 wt. % niobium, 0-3 wt. % silicon. As defined herein, a standard titanium alloy includes 80-99 wt. % titanium, 0-6 wt. % aluminum, 0-3 wt. % tin, 0-1 wt. % palladium, 0-8 wt. % vanadium, 0-15 wt. % molybdenum, 0-1 wt. % nickel, 0-0.3 wt. % ruthenium, 0-6 wt. % chromium, 0-4 wt. % zirconium, 0-4 wt. % niobium, 0-1 wt. % silicon, 0.0.5 wt. % cobalt, 0-2 wt. % iron. As defined herein, a standard tungsten alloy includes 85-98 wt. % tungsten, 0-8 wt. % nickel, 0-5 wt. % copper, 0-5 wt. % molybdenum, 0-4 wt. % iron. As defined herein, a standard molybdenum alloy includes 90-99.5 wt. % molybdenum, 0-1 wt. % nickel, 0-1 wt. % titanium, 0-1 wt. % zirconium, 0-30 wt. % tungsten, 0-2 wt. % hafnium, 0-2 wt. % lanthanum. As defined herein, a standard copper alloy includes 55-95 wt. % copper, 0-40 wt. % zinc, 0-10 wt. % tin, 0-10 wt. % lead, 0-1 wt. % iron, 0-5 wt. % silicon, 0-12 wt. % manganese, 0-12 wt. % aluminum, 0-3 wt. % beryllium, 0-1 wt. % cobalt, 0-20 wt. % nickel. As defined herein, a standard beryllium-copper alloy includes 95-98.5 wt. % copper, 1-4 wt. % beryllium, 0-1 wt. % cobalt, and 0-0.5 wt. % silicon. As defined herein, a standard titanium-nickel alloy (e.g., Nitinol alloy) includes 42-58 wt. % nickel and 42-58 wt. % titanium. The rhenium effect has been found to occur when the atomic weight of rhenium in the metal alloy is at least 15% (e.g., 15-99 awt. % rhenium in the metal alloy and all values and ranges therebetween). For example, for standard stainless-steel alloys, the rhenium effect can begin to be present when the stainless steel alloy is modified to include a rhenium amount of at least 5-10 wt. % (and all values and ranges therebetween) of the stainless steel alloy. For standard CoCr alloys, the rhenium effect can begin to be present when the CoCr alloy is modified to include a rhenium amount of at least 4.8-9.5 wt. % (and all values and ranges therebetween) of the CoCr alloy. For standard TiAlV alloys, the rhenium effect can begin to be present when the TiAlV alloy is modified to include a rhenium amount of at least 4.5-9 wt. % (and all values and ranges therebetween) of the TiAlV alloy. It can be appreciated, the rhenium content in the above non-limiting examples can be greater than the minimum amount to create the rhenium effect in the metal alloy.

In accordance with another and/or alternative aspect of the present disclosure, the metal alloy that is used to partially or fully form the expandable medical device includes at least 5 awt. % (e.g., 5-99 awt. % and all values and ranges therebetween) rhenium, and 0.1-96 wt. % (and all values and ranges therebetween) of one or more additives selected from the group of aluminum, boron, beryllium, bismuth, cadmium, calcium, cerium, chromium, cobalt, copper, gallium, gold, hafnium, iridium, iron, lanthanum, lanthanum oxide, lead, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other metals (e.g., metals other than additives), carbon, oxygen, phosphorous, sulfur, hydrogen and/or nitrogen, and which metal alloy exhibits a rhenium effect. In one non-limiting embodiment, the metal alloy that is used to partially or fully form the expandable medical device is a standard stainless steel alloy that has been modified to include at least 15 awt. % rhenium. In another non-limiting embodiment, the metal alloy that is used to partially or fully form the expandable medical device is a standard cobalt chromium alloy that has been modified to include at least 15 awt. % rhenium. In another non-limiting embodiment, the metal alloy that is used to partially or fully form the expandable medical device is a standard TiAlV alloy that has been modified to include at least 15 awt. % rhenium. In another non-limiting embodiment, the metal alloy that is used to partially or fully form the expandable medical device is a standard aluminum alloy that has been modified to include at least 15 awt. % rhenium. In another non-limiting embodiment, the metal alloy that is used to partially or fully form the expandable medical device is a standard nickel alloy that has been modified to include at least 15 awt. % rhenium. In another non-limiting embodiment, the metal alloy that is used to partially or fully form the expandable medical device is a standard titanium alloy that has been modified to include at least 15 awt. % rhenium. In another non-limiting embodiment, the metal alloy that is used to partially or fully form the expandable medical device is a standard tungsten alloy that has been modified to include at least 15 awt. % rhenium. In another non-limiting embodiment, the metal alloy that is used to partially or fully form the expandable medical device is a standard molybdenum alloy that has been modified to include at least 15 awt. % rhenium. In another non-limiting embodiment, the metal alloy that is used to partially or fully form the expandable medical device is a standard copper alloy that has been modified to include at least 15 awt. % rhenium. In another non-limiting embodiment, the metal alloy that is used to partially or fully form the expandable medical device is a standard beryllium-copper alloy that has been modified to include at least 15 awt. % rhenium.

In accordance with another and/or alternative aspect of the present disclosure, the metal alloy that is used to partially or fully form the expandable medical device includes rhenium and molybdenum, and the weight percent of rhenium in the metal alloy is optionally greater than the weight percent of molybdenum in the metal alloy, and the weight percent of one or more additive (e.g., aluminum, boron, beryllium, bismuth, cadmium, calcium, cerium, chromium, cobalt, copper, gallium, gold, hafnium, iridium, iron, lanthanum, lanthanum oxide, lead, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium) in the metal alloy is optionally greater that the weight percent of molybdenum in the metal alloy, and the metal alloy optionally includes 0-2 wt. % of a combination of other metals (metals other than the additive), carbon, oxygen, phosphorous, sulfur, hydrogen and/or nitrogen. In one non-limiting embodiment, the metal alloy that is used to partially or fully form the expandable medical device includes rhenium and molybdenum, and the weight percent of rhenium plus the combined weight percent of additives is greater than the weight percent of molybdenum, and the metal alloy optionally includes 0-2 wt. % of a combination of other metals (metals other than the additive), carbon, oxygen, phosphorous, sulfur, hydrogen and/or nitrogen.

In accordance with another and/or alternative aspect of the present disclosure, the metal alloy that is used to partially or fully form the expandable medical device includes rhenium and molybdenum, and the atomic weight percent of rhenium to the atomic weight percent of the combination of one or more of bismuth, niobium, tantalum, tungsten, titanium, vanadium, chromium, manganese, yttrium, zirconium, technetium, ruthenium, rhodium, hafnium, osmium, copper, and iridium is 0.4:1 to 2.5:1 (and all values and ranges therebetween).

In accordance with another and/or alternative aspect of the present disclosure, the metal alloy that is used to partially or fully form the expandable medical device includes at least 5 awt. % (e.g., 5-99 awt. % and all values and ranges therebetween) rhenium plus at least two metals selected from the group of molybdenum, bismuth, chromium, iridium, niobium, tantalum, titanium, yttrium, and zirconium, and the content of the metal alloy that includes other elements and compounds is 0-0.1 wt. %. In another non-limiting embodiment, the metal alloy includes rhenium, molybdenum, and chromium. In another non-limiting embodiment, the metal alloy includes at least 35 wt. % (e.g., 35-75 wt. % and all values and ranges therebetween) rhenium, and the metal alloy also includes chromium. In one non-limiting embodiment, the metal alloy includes at least 35 wt. % rhenium and at least 25 wt. % (e.g., 25-49.9 wt. % and all values and ranges therebetween) of the metal alloy includes chromium, and optionally 0.1-40 wt. % (and all values and ranges therebetween) of the metal alloy includes one or more of aluminum, boron, beryllium, bismuth, cadmium, calcium, cerium, chromium, cobalt, copper, gallium, gold, hafnium, iridium, iron, lanthanum, lanthanum oxide, lead, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other metals, carbon, oxygen, phosphorous, sulfur, hydrogen and/or nitrogen. In another non-limiting embodiment, the metal alloy includes 15-50 awt. % rhenium (and all values and ranges therebetween) and 0.5-70 awt. % chromium (and all values and ranges therebetween). In another non-limiting embodiment, the metal alloy includes 15-50 awt. % rhenium (and all values and ranges therebetween) and 0.5-70 awt. % tantalum (and all values and ranges therebetween). In another non-limiting embodiment, the metal alloy includes 15-50 awt. % rhenium (and all values and ranges therebetween) and 0.5-70 awt. % niobium (and all values and ranges therebetween). In another non-limiting embodiment, the metal alloy includes 15-50 awt. % rhenium (and all values and ranges therebetween) and 0.5-70 awt. % titanium (and all values and ranges therebetween). In another non-limiting embodiment, the metal alloy includes 15-50 awt. % rhenium (and all values and ranges therebetween) and 0.5-70 awt. % zirconium (and all values and ranges therebetween). In another non-limiting embodiment, the metal alloy includes 15-50 awt. % rhenium (and all values and ranges therebetween) and 0.5-70 awt. % molybdenum (and all values and ranges therebetween). In another non-limiting embodiment, the metal alloy includes at least 15 awt. % rhenium, greater than 50 wt. % titanium (e.g., 51-80 wt. % and all values and ranges therebetween), 15-45 wt. % (and all values and ranges therebetween) niobium, 0-10 wt. % (and all values and ranges therebetween) zirconium, 0-15 wt. % (and all values and ranges therebetween) tantalum, and 0-8 wt. % molybdenum (and all values and ranges therebetween).

Several non-limiting examples of metal alloys that can be used to partially or fully form the orthopedic medical device are set forth below in weight percent:

| Component/Wt. % | Ex. 1 | Ex. 2 | Ex. 3 | Ex. 4 |
|---|---|---|---|---|
| Al | 0-35% | 0-30% | 0-25% | 0-10% |
| Bi | 0-20% | 0-20% | 0-20% | 0-20% |
| Cr | 0-60% | 0-35% | 0-30% | 0-25% |
| Co | 0-60% | 0-50% | 0-40% | 0-20% |
| Mo | 0-95% | 0-80% | 0-55% | 0-30% |
| Nb | 0-80% | 0-60% | 0-50% | 0-20% |
| Ni | 0-60% | 0-55% | 0-40% | 0-20% |
| Re | 0.1-70% | 4.5-70% | 5-70% | 5-70% |
| Ta | 0-80% | 0-50% | 0-40% | 0-25% |
| Ti | 0-60% | 0-55% | 0-40% | 0-20% |
| V | 0-20% | 0-15% | 0-10% | 0-10% |
| W | 0-80% | 0-70% | 0-50% | 0-20% |
| Y | 0-20% | 0-15% | 0-10% | 0-10% |
| Zr | 0-20% | 0-15% | 0-10% | 0-10% |

| Component/Wt. % | Ex. 5 | Ex. 6 | Ex. 7 | Ex. 8 |
|---|---|---|---|---|
| Ag | 0-20% | 0-20% | 0-20% | 0-20% |
| Al | 0-35% | 0-30% | 5-30% | 0-25% |
| Bi | 0-20% | 0-20% | 0-20% | 0-20% |
| Cr | 10-40% | 0-40% | 0-40% | 0-40% |
| Cu | 0-20% | 0-20% | 0-20% | 0-20% |
| Co | 10-60% | 0-60% | 0-60% | 0-60% |
| Fe | 0-80% | 30-80% | 0-80% | 0-70% |
| Hf | 0-20% | 0-20% | 0-20% | 0-20% |
| Ir | 0-20% | 0-20% | 0-20% | 0-20% |
| Mg | 0-20% | 0-20% | 0-20% | 0-20% |
| Mn | 0-20% | 0-40% | 0-20% | 0-20% |
| Mo | 0-60% | 0-60% | 0-80% | 0-70% |
| Nb | 0-60% | 0-60% | 0-65% | 20-60% |
| Ni | 0-60% | 5-55% | 0-52% | 0-50% |
| Os | 0-20% | 0-20% | 0-20% | 0-20% |
| Pt | 0-20% | 0-20% | 0-20% | 0-20% |
| Re | 4.5-98% | 4.5-90% | 4.5-80% | 4.5-70% |
| Rh | 0-20% | 0-20% | 0-20% | 0-20% |
| Si | 0-20% | 0-20% | 0-20% | 0-20% |
| Sn | 0-20% | 0-20% | 0-20% | 0-20% |
| Ta | 0-60% | 0-60% | 5-65% | 0-60% |
| Tc | 0-20% | 0-20% | 0-20% | 0-20% |
| Ti | 0-60% | 0-55% | 0-53% | 0-50% |
| V | 0-20% | 0-20% | 2-20% | 0-20% |
| W | 0-60% | 0-60% | 0-80% | 0-70% |
| Y | 0-20% | 0-20% | 0-20% | 0-20% |
| Zr | 0-20% | 0-20% | 0-20% | 5-20% |

| Component/Wt. % | Ex. 9 | Ex. 10 | Ex. 11 | Ex. 12 |
|---|---|---|---|---|
| Ag | 0-5% | 0-5% | 0-5% | 0-5% |
| Al | 0-5% | 0-5% | 1-15% | 0-20% |
| Bi | 0-5% | 0-5% | 0-5% | 0-5% |
| Cr | 1-28% | 1-30% | 0-5% | 0-30% |
| Cu | 0-20% | 0-5% | 0-5% | 0-25% |
| Co | 0-5% | 1-60% | 0-5% | 0-60% |
| Fe | 10-80% | 0-25% | 0-5% | 0-80% |
| Hf | 0-5% | 0-5% | 0-5% | 0-5% |
| Ir | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-8% | 0-25% | 0-5% | 0-98% |
| Nb | 0-5% | 0-5% | 0-5% | 0-95% |
| Ni | 1-20% | 1-45% | 0-5% | 0-50% |
| Os | 0-5% | 0-5% | 0-5% | 0-5% |
| Pt | 0-5% | 0-5% | 0-5% | 0-5% |
| Re | 5-20% | 4.8-20% | 4.5-20% | 4.5-20% |
| Rh | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 0-5% | 0-98% |
| Tc | 0-5% | 0-5% | 0-5% | 0-5% |
| Ti | 0-5% | 0-5% | 40-93% | 0-93% |
| V | 0-5% | 0-5% | 1-10% | 0-20% |
| W | 0-5% | 0-20% | 0-5% | 0-98% |
| Y | 0-5% | 0-5% | 0-5% | 0-5% |
| Zr | 0-5% | 0-5% | 0-5% | 0-5% |

| Component/Wt. % | Ex. 13 | Ex. 14 | Ex. 15 | Ex. 16 |
|---|---|---|---|---|
| Mo | 30-80% | 35-80% | 30-70% | 35-65% |
| Hf | 0.8-1.4% | 0-2% | 0-2.5% | 0-2.5% |
| Re | 7-49% | 7-49% | 7-60% | 7.5-49% |
| Ta | 0-2% | 0-2% | 0-50% | 0-50% |
| W | 0-2% | 0-2% | 0-50% | 20-50% |

| Component/Wt. % | Ex. 17 | Ex. 18 | Ex. 10 | Ex. 20 |
|---|---|---|---|---|
| W | 20-93% | 60-92% | 20-75% | 5-98% |
| Re | 6-60% | 8-40% | 7.5-47.5% | 0-80% |
| Mo | 0-47.5% | <0.5% | 1-47.5% | 0-80% |

| Component/Wt. % | Ex. 21 | Ex. 22 | Ex. 23 | Ex. 24 |
|---|---|---|---|---|
| Re | 5-60% | 5-60% | 5-60% | 5-60% |
| Mo | 0-55% | 10-55% | 10-55% | 10-55% |
| Bi | 1-42 | 0-32 | 0-32 | 0-32 |
| Cr | 0-32 | 1-42 | 0-32 | 0-32 |
| Ir | 0-32 | 0-32 | 1-42 | 0-32 |
| Nb | 0-32 | 0-32 | 0-32 | 1-42 |
| Ta | 0-32 | 0-32 | 0-32 | 0-32 |
| Ti | 0-32 | 0-32 | 0-32 | 0-32 |
| Y | 0-32 | 0-32 | 0-32 | 0-32 |
| Zr | 0-32 | 0-32 | 0-32 | 0-32 |
| Component/Wt. % | Ex. 25 | Ex. 26 | Ex. 27 | Ex. 28 |
| Re | 5-60% | 5-60% | 5-60% | 5-60% |
| Mo | 15-55% | 15-55% | 15-55% | 15-55% |
| Bi | 0-32 | 0-32 | 0-32 | 0-32 |
| Cr | 0-32 | 0-32 | 0-32 | 0-32 |
| Ir | 0-32 | 0-32 | 0-32 | 0-32 |
| Nb | 0-32 | 0-32 | 0-32 | 0-32 |
| Ta | 1-42 | 0-32 | 0-32 | 0-32 |
| Ti | 0-32 | 1-42 | 0-32 | 0-32 |
| Y | 0-32 | 0-32 | 1-42 | 0-32 |
| Zr | 0-32 | 0-32 | 0-32 | 1-42 |
| Component/Wt. % | Ex. 29 | Ex. 30 | Ex. 31 | Ex. 32 |
| Re | 50-75% | 55-75% | 60-75% | 65-75% |
| Cr | 25-50% | 25-45% | 25-40% | 25-35% |
| Mo | 0-25% | 0-25% | 0-25% | 0-25% |
| Bi | 0-25% | 0-25% | 0-25% | 0-25% |
| Ir | 0-25% | 0-25% | 0-25% | 0-25% |
| Nb | 0-25% | 0-25% | 0-25% | 0-25% |
| Ta | 0-25% | 0-25% | 0-25% | 0-25% |
| V | 0-25% | 0-25% | 0-25% | 0-25% |
| W | 0-25% | 0-25% | 0-25% | 0-25% |
| Mn | 0-25% | 0-25% | 0-25% | 0-25% |
| Tc | 0-25% | 0-25% | 0-25% | 0-25% |
| Ru | 0-25% | 0-25% | 0-25% | 0-25% |
| Rh | 0-25% | 0-25% | 0-25% | 0-25% |
| Hf | 0-25% | 0-25% | 0-25% | 0-25% |
| Os | 0-25% | 0-25% | 0-25% | 0-25% |
| Cu | 0-25% | 0-25% | 0-25% | 0-25% |
| Ir | 0-25% | 0-25% | 0-25% | 0-25% |
| Ti | 0-25% | 0-25% | 0-25% | 0-25% |
| Y | 0-25% | 0-25% | 0-25% | 0-25% |
| Zr | 0-25% | 0-25% | 0-25% | 0-25% |
| Ag | 0-25% | 0-25% | 0-25% | 0-25% |
| Al | 0-25% | 0-25% | 0-25% | 0-22% |
| Co | 0-25% | 0-25% | 0-25% | 0-25% |
| Fe | 0-25% | 0-25% | 0-25% | 0-25% |
| Mg | 0-25% | 0-25% | 0-25% | 0-25% |
| Ni | 0-25% | 0-25% | 0-25% | 0-25% |
| Pt | 0-25% | 0-25% | 0-25% | 0-25% |
| Si | 0-25% | 0-25% | 0-25% | 0-25% |
| Sn | 0-25% | 0-25% | 0-25% | 0-25% |
| Component/Wt. % | Ex. 33 | Ex. 34 | Ex. 35 | Ex. 36 |
| Re | 50-75% | 55-72% | 60-70% | 62-70% |
| Cr | 24-49% | 27-44% | 29-39% | 29-37% |
| Mo | 1-15% | 1-10% | 1-8% | 1-5% |
| Bi | 0-15% | 0-10% | 0-8% | 0-5% |
| Ir | 0-15% | 0-10% | 0-8% | 0-5% |
| Nb | 0-15% | 0-10% | 0-8% | 0-5% |
| Ta | 0-15% | 0-10% | 0-8% | 0-5% |
| V | 0-15% | 0-10% | 0-8% | 0-5% |
| W | 0-15% | 0-10% | 0-8% | 0-5% |
| Mn | 0-15% | 0-10% | 0-8% | 0-5% |
| Tc | 0-15% | 0-10% | 0-8% | 0-5% |
| Ru | 0-15% | 0-10% | 0-8% | 0-5% |
| Rh | 0-15% | 0-10% | 0-8% | 0-5% |
| Hf | 0-15% | 0-10% | 0-8% | 0-5% |
| Os | 0-15% | 0-10% | 0-8% | 0-5% |
| Cu | 0-15% | 0-10% | 0-8% | 0-5% |
| Ir | 0-15% | 0-10% | 0-8% | 0-5% |
| Ti | 0-15% | 0-10% | 0-8% | 0-5% |
| Y | 0-15% | 0-10% | 0-8% | 0-5% |
| Zr | 0-15% | 0-10% | 0-8% | 0-5% |
| Ag | 0-15% | 0-10% | 0-8% | 0-5% |
| Al | 0-15% | 0-10% | 0-8% | 0-5% |
| Co | 0-15% | 0-10% | 0-8% | 0-5% |
| Fe | 0-15% | 0-10% | 0-8% | 0-5% |
| Mg | 0-15% | 0-10% | 0-8% | 0-5% |
| Ni | 0-15% | 0-10% | 0-8% | 0-5% |
| Pt | 0-15% | 0-10% | 0-8% | 0-5% |
| Si | 0-15% | 0-10% | 0-8% | 0-5% |
| Sn | 0-15% | 0-10% | 0-8% | 0-5% |

| Component/Wt. % | Ex. 37 | Ex. 38 | Ex. 39 | Ex. 40 |
|---|---|---|---|---|
| Mo | 40-95% | 40-95% | 40-95% | 40-95% |
| Co | ≤0.002% | ≤0.002% | ≤0.002% | ≤0.002% |
| Fe | ≤0.02% | ≤0.02% | ≤0.02% | ≤0.02% |
| Hf | 0.1-2.5% | 0-2.5% | 0-2.5% | 0-2.5% |
| Os | ≤1% | ≤1% | ≤1% | ≤1% |
| Nb | ≤0.01% | ≤0.01% | ≤0.01% | ≤0.01% |
| Pt | ≤1% | ≤1% | ≤1% | ≤1% |
| Re | 5-49% | 5-49% | 5-49% | 5-49% |
| Sn | ≤0.002% | ≤0.002% | ≤0.002% | ≤0.002% |
| Ta | 0-50% | 0-50% | 0-50% | 0-50% |
| Tc | ≤1% | ≤1% | ≤1% | ≤1% |
| Ti | ≤1% | ≤1% | ≤1% | ≤1% |
| V | ≤1% | ≤1% | ≤1% | ≤1% |
| W | 0-50% | 0-50% | 0-50% | 0.5-50% |
| Zr | ≤1% | ≤1% | ≤1% | ≤1% |
| Ag | 0-5% | 0-5% | 0-5% | 0-5% |
| Al | 0-5% | 0-5% | 0-5% | 0-5% |
| Co | 0-5% | 0-5% | 0-5% | 0-5% |
| Mg | 0-5% | 0-5% | 0-5% | 0-5% |
| Ni | 0-5% | 0-5% | 0-5% | 0-5% |
| Si | 0-5% | 0-5% | 0-5% | 0-5% |
| Sn | 0-5% | 0-5% | 0-5% | 0-5% |

| Component/Wt. % | Ex. 41 | Ex. 42 | Ex. 43 |
|---|---|---|---|
| W | 20-95% | 60-95% | 20-80% |
| Re | 5-47.5% | 5-40% | 5-47.5% |
| Mo | 0-47.5% | <0.5% | 1-47.5% |
| Cu | <0.5% | <0.5% | <0.5% |
| Co | ≤0.002% | ≤0.002% | ≤0.002% |
| Fe | ≤0.02% | ≤0.02% | ≤0.02% |
| Hf | <0.5% | <0.5% | <0.5% |
| Os | <0.5% | <0.5% | <0.5% |
| Nb | ≤0.01% | ≤0.01% | ≤0.01% |
| Pt | <0.5% | <0.5% | <0.5% |
| Sn | ≤0.002% | ≤0.002% | ≤0.002% |
| Ta | <0.5% | <0.5% | <0.5% |
| Tc | <0.5% | <0.5% | <0.5% |
| Ti | <0.5% | <0.5% | <0.5% |
| V | <0.5% | <0.5% | <0.5% |
| Zr | <0.5% | <0.5% | <0.5% |
| Ag | 0-5% | 0-5% | 0-5% |
| Al | 0-5% | 0-5% | 0-5% |
| Fe | 0-5% | 0-5% | 0-5% |
| Mg | 0-5% | 0-5% | 0-5% |
| Ni | 0-5% | 0-5% | 0-5% |
| Si | 0-5% | 0-5% | 0-5% |

| Component/Wt. % | Ex. 44 | Ex. 45 | Ex. 46 | Ex. 47 |
|---|---|---|---|---|
| W | 1-94.9% | 1-94.9% | 1-94.9% | 10-95% |
| Cu | 0.1-94% | 0.1-94% | 0.1-94% | 1-84% |
| Co | ≤0.002% | ≤0.002% | ≤0.002% | ≤0.002% |
| Fe | ≤0.02% | ≤0.02% | ≤0.02% | ≤0.02% |
| Hf | 0.1-2.5% | 0-2.5% | 0-2.5% | 0-2.5% |
| Os | ≤1% | ≤1% | ≤1% | ≤1% |
| Mo | 0-5% | 0.1-3% | 0-2% | 0-3% |
| Nb | ≤0.01% | ≤0.01% | ≤0.01% | ≤0.01% |
| Pt | ≤1% | ≤1% | ≤1% | ≤1% |
| Re | 5-40% | 5-40% | 5-40% | 6-40% |
| Sn | ≤0.002% | ≤0.002% | ≤0.002% | ≤0.002% |
| Ta | 0-50% | 0-50% | 0-50% | 0-50% |
| Tc | ≤1% | ≤1% | ≤1% | ≤1% |
| Ti | ≤1% | ≤1% | ≤1% | ≤1% |
| V | ≤1% | ≤1% | ≤1% | ≤1% |
| Zr | ≤1% | ≤1% | ≤1% | ≤1% |
| Ag | 0-5% | 0-5% | 0-5% | 0-5% |
| Al | 0-5% | 0-5% | 0-5% | 0-5% |
| Fe | 0-5% | 0-5% | 0-5% | 0-5% |
| Mg | 0-5% | 0-5% | 0-5% | 0-5% |
| Ni | 0-5% | 0-5% | 0-5% | 0-5% |
| Si | 0-5% | 0-5% | 0-5% | 0-5% |

| Component/Wt. % | Ex. 48 | Ex. 49 | Ex. 50 |
|---|---|---|---|
| W | 20-96% | 25-92% | 30-88% |
| Cu | 2-74% | 2-68% | 5-62% |
| Co | ≤0.002% | ≤0.002% | ≤0.002% |
| Hf | 0-2.5% | 0-2.5% | 0-2.5% |
| Os | ≤1% | ≤1% | ≤1% |
| Mo | 0-3% | 0-2% | 0-1% |
| Nb | ≤0.01% | ≤0.01% | ≤0.01% |
| Pt | ≤1% | ≤1% | ≤1% |
| Re | 6-40% | 7-40% | 8-40% |
| Sn | ≤0.002% | ≤0.002% | ≤0.002% |
| Ta | 0-50% | 0.5-50% | 0-50% |
| Tc | ≤1% | ≤1% | ≤1% |
| Ti | ≤1% | ≤1% | ≤1% |
| V | ≤1% | ≤1% | ≤1% |
| Ag | 0-5% | 0-5% | 0-5% |
| Al | 0-5% | 0-5% | 0-5% |
| Fe | 0-5% | 0-5% | 0-5% |
| Mg | 0-5% | 0-5% | 0-5% |
| Ni | 0-5% | 0-5% | 0-5% |
| Si | 0-5% | 0-5% | 0-5% |

| Component/Wt. % | Ex. 51 | Ex. 52 | Ex. 53 | Ex. 54 |
|---|---|---|---|---|
| W | 25-88% | 35-87% | 40-86% | 50-85% |
| Cu | 5-68% | 5-57% | 5-51% | 5-40% |
| Hf | 0.8-1.4% | 0-2.5% | 0-2.5% | 0-2.5% |
| Re | 0-40% | 0-40% | 0-40% | 0-40% |
| Ta | 0-50% | 0-50% | 0-50% | 0-50% |

| Component/Wt. % | Ex. 55 | Ex. 56 | Ex. 57 |
|---|---|---|---|
| Ti | 55-66% | 65-76% | 70-76% |
| Mo | 20-41% | 20-31% | 20-26% |
| Re | 4-20% | 4-20% | 4-20% |
| Yt | <0.5% | <0.5% | <0.5% |
| Nb | <0.5% | <0.5% | <0.5% |
| Co | <0.5% | <0.5% | <0.5% |
| Cr | <0.5% | <0.5% | <0.5% |
| Zr | <0.5% | <0.5% | <0.5% |

| Component/Wt. % | Ex. 58 | Ex. 59 | Ex. 60 |
|---|---|---|---|
| W | 20-95% | 60-93% | 20-80% |
| Re | 5-47.5% | 7-40% | 5-47.5% |
| Mo | 0-47.5% | <0.5% | 1-47.5% |
| Cu | <0.5% | <0.5% | <0.5% |
| Co | ≤0.002% | <0.002% | ≤0.002% |
| Fe | ≤0.02% | ≤0.02% | ≤0.02% |
| Hf | <0.5% | <0.5% | <0.5% |
| Os | <0.5% | <0.5% | <0.5% |
| Nb | ≤0.01% | ≤0.01% | ≤0.01% |
| Pt | <0.5% | <0.5% | <0.5% |
| Sn | ≤0.002% | ≤0.002% | ≤0.002% |
| Ta | <0.5% | <0.5% | <0.5% |
| Tc | <0.5% | <0.5% | <0.5% |
| Ti | <0.5% | <0.5% | <0.5% |
| V | <0.5% | <0.5% | <0.5% |
| Zr | <0.5% | <0.5% | <0.5% |
| Ag | 0-5% | 0-5% | 0-5% |
| Al | 0-5% | 0-5% | 0-5% |
| Fe | 0-5% | 0-5% | 0-5% |
| Mg | 0-5% | 0-5% | 0-5% |
| Ni | 0-5% | 0-5% | 0-5% |
| Si | 0-5% | 0-5% | 0-5% |

| Component/Wt. % | Ex. 61 | Ex. 62 | Ex. 63 | Ex. 64 |
|---|---|---|---|---|
| Ag | 0-10% | 0-10% | 0-10% | 0-10% |
| Al | 0-10% | 0-10% | 0-10% | 2-10% |
| B | 0-10% | 0-10% | 0-10% | 0-10% |
| Bi | 0-10% | 0-10% | 0-10% | 0-10% |
| Cr | 2-30% | 10-30% | 0-20% | 0-20% |
| Cu | 0-10% | 0-10% | 0-10% | 0-10% |
| Co | 0-10% | 32-70% | 0-10% | 0-10% |
| Fe | 50-80% | 0-20% | 0-10% | 0-10% |
| Hf | 0-10% | 0-10% | 0-10% | 0-10% |
| Ir | 0-10% | 0-10% | 0-10% | 0-10% |
| La | 0-10% | 0-10% | 0-10% | 0-10% |
| Mg | 0-10% | 0-10% | 0-10% | 0-10% |
| Mn | 0-20% | 0-10% | 0-10% | 0-10% |
| Mo | 0-10% | 0-30% | 0-16% | 0-16% |
| Nb | 0-10% | 0-10% | 0-10% | 0-10% |
| Ni | 0.1-30% | 0.1-40% | 0-10% | 0-10% |
| Os | 0-10% | 0-10% | 0-10% | 0-10% |
| Pt | 0-10% | 0-10% | 0-10% | 0-10% |
| Re | 5-40% | 4.8-40% | 4.5-80% | 4.5-80% |
| Rh | 0-10% | 0-10% | 0-10% | 0-10% |
| Se | 0-10% | 0-10% | 0-10% | 0-10% |
| Si | 0-10% | 0-10% | 0-10% | 0-10% |
| Sn | 0-10% | 0-10% | 0-12% | 0-12% |
| Ta | 0-10% | 0-10% | 0-10% | 0-10% |
| Tc | 0-10% | 0-10% | 0-10% | 0-10% |
| Ti | 0-10% | 0-10% | 70-91.5% | 70-91.5% |
| V | 0-10% | 0-10% | 0-10% | 0.01-10% |
| W | 0-10% | 0-20% | 0-10% | 0-10% |
| Y | 0-10% | 0-10% | 0-10% | 0-10% |
| Zr | 0-10% | 0-10% | 0-10% | 0-10% |

| Component/Wt. % | Ex. 65 | Ex. 66 | Ex. 67 | Ex. 68 |
|---|---|---|---|---|
| Ag | 0-10% | 0-10% | 0-10% | 0-10% |
| Al | 0-10% | 0-10% | 0-10% | 0-10% |
| B | 0-10% | 0-10% | 0-10% | 0-10% |
| Bi | 0-10% | 0-10% | 0-10% | 0-10% |
| Cr | 0-10% | 0-20% | 0-20% | 0-10% |
| Cu | 0-10% | 0-10% | 0-50% | 0-10% |
| Co | 0-10% | 0-10% | 0-10% | 0-10% |
| Fe | 0-10% | 0-10% | 0-10% | 0-10% |
| Hf | 0-10% | 0-10% | 0-10% | 0-10% |
| Ir | 0-10% | 0-10% | 0-10% | 0-12% |
| La | 0-10% | 0-10% | 0-10% | 0-10% |
| Mg | 0-10% | 0-10% | 0-10% | 0-10% |
| Mn | 0-10% | 0-10% | 0-10% | 0-10% |
| Mo | 0-55% | 40-93% | 0-50% | 0-20% |
| Nb | 0-10% | 0-10% | 0-10% | 40-85% |
| Ni | 0-45% | 0-10% | 0-10% | 0-10% |
| Os | 0-10% | 0-10% | 0-10% | 0-10% |
| Pt | 0-10% | 0-10% | 0-10% | 0-10% |
| Re | 14-40% | 7-40% | 7-40% | 7-40% |
| Rh | 0-10% | 0-10% | 0-10% | 0-10% |
| Se | 0-10% | 0-10% | 0-10% | 0-10% |
| Si | 0-10% | 0-10% | 0-10% | 0-10% |
| Sn | 0-10% | 0-10% | 0-10% | 0-10% |
| Ta | 35-84% | 0-50% | 0-50% | 0-35% |
| Tc | 0-10% | 0-10% | 0-10% | 0-10% |
| Ti | 0-10% | 0-10% | 0-10% | 0-10% |
| V | 0-10% | 0-10% | 0-10% | 0-10% |
| W | 0.1-25% | 0-50% | 14-10% | 0-15% |
| Y | 0-10% | 0-10% | 0-10% | 0-10% |
| Zr | 0-10% | 0-10% | 0-50% | 0-10% |

| Component/Wt. % | Ex. 69 | Ex. 70 | Ex. 71 | Ex. 72 |
|---|---|---|---|---|
| Ag | 0-10% | 0-10% | 0-5% | 0-5% |
| Al | 0-10% | 0-10% | 0-5% | 5-7% |
| B | 0-10% | 0-10% | 0-5% | 0-5% |
| Bi | 0-10% | 0-10% | 0-5% | 0-5% |
| Cr | 0-10% | 1-95% | 12-28% | 0-5% |
| Cu | 0-10% | 0-10% | 0-5% | 0-5% |
| Co | 0-10% | 0-10% | 36-68% | 0-5% |
| Fe | 0-10% | 0-10% | 0-18% | 0-5% |
| Hf | 0-10% | 0-10% | 0-5% | 0-5% |
| Ir | 0-10% | 0-10% | 0-5% | 0-5% |
| La | 0-10% | 0-10% | 0-5% | 0-5% |
| Mg | 0-10% | 0-10% | 0-5% | 0-5% |
| Mn | 0-10% | 0-10% | 0-5% | 0-5% |
| Mo | 0-10% | 0-20% | 0-12% | 0-5% |
| Nb | 0-10% | 0-10% | 0-5% | 0-5% |
| Ni | 30-58% | 0-10% | 9-36% | 0-5% |
| Os | 0-10% | 0-10% | 0-5% | 0-5% |
| Pt | 0-10% | 0-10% | 0-5% | 0-5% |
| Re | 5-40% | 5-40% | 4.8-40% | 4.5-40% |
| Rh | 0-10% | 0-10% | 0-5% | 0-5% |
| Se | 0-10% | 0-10% | 0-5% | 0-5% |
| Si | 0-10% | 0-10% | 0-5% | 0-5% |
| Sn | 0-10% | 0-10% | 0-5% | 0-5% |
| Ta | 0-10% | 0-10% | 0-5% | 0-5% |
| Tc | 0-10% | 0-10% | 0-5% | 0-5% |
| Ti | 30-58% | 0-40% | 0-5% | 70-91.5% |
| V | 0-10% | 0-10% | 0-5% | 3-6% |
| W | 0-10% | 0-10% | 0-16% | 0-5% |
| Y | 0-10% | 0-10% | 0-5% | 0-5% |
| Zr | 0-10% | 0-20% | 0-5% | 0-5% |

| Component/Wt. % | Ex. 73 | Ex. 74 | Ex. 75 | Ex. 76 |
|---|---|---|---|---|
| Ag | 0-8% | 0-8% | 0-8% | 0-8% |
| Al | 0-8% | 0-8% | 0-8% | 2-10% |
| B | 0-8% | 0-8% | 0-8% | 0-8% |
| Bi | 0-8% | 0-8% | 0-8% | 0-8% |
| Cr | 2-30% | 10-30% | 0-20% | 0-20% |
| Cu | 0-8% | 0-8% | 0-8% | 0-8% |
| Co | 0-8% | 32-70% | 0-8% | 0-8% |
| Fe | 50-80% | 0-20% | 0-8% | 0-8% |
| Hf | 0-8% | 0-8% | 0-8% | 0-8% |
| Ir | 0-8% | 0-8% | 0-8% | 0-8% |
| La | 0-8% | 0-8% | 0-8% | 0-8% |
| Mg | 0-8% | 0-8% | 0-8% | 0-8% |
| Mn | 0-20% | 0-8% | 0-8% | 0-8% |
| Mo | 0-8% | 0-30% | 0-16% | 0-16% |
| Nb | 0-8% | 0-8% | 0-8% | 0-8% |
| Ni | 0.1-30% | 0.1-40% | 0-8% | 0-8% |
| Os | 0-8% | 0-8% | 0-8% | 0-8% |
| Pt | 0-8% | 0-8% | 0-8% | 0-8% |
| Re | 5-40% | 4.8-40% | 4.5-80% | 4.5-80% |
| Rh | 0-8% | 0-8% | 0-8% | 0-8% |
| Se | 0-8% | 0-8% | 0-8% | 0-8% |
| Si | 0-8% | 0-8% | 0-8% | 0-8% |
| Sn | 0-8% | 0-8% | 0-12% | 0-12% |
| Ta | 0-8% | 0-8% | 0-8% | 0-8% |
| Tc | 0-8% | 0-8% | 0-8% | 0-8% |
| Ti | 0-8% | 0-8% | 70-91.5% | 70-91.5% |
| V | 0-8% | 0-8% | 0-8% | 0.01-10% |
| W | 0-8% | 0-20% | 0-8% | 0-8% |
| Y | 0-8% | 0-8% | 0-8% | 0-8% |
| Zr | 0-8% | 0-8% | 0-8% | 0-8% |

| Component/Wt. % | Ex. 77 | Ex. 78 | Ex. 79 | Ex. 80 |
|---|---|---|---|---|
| Ag | 0-8% | 0-8% | 0-8% | 0-8% |
| Al | 0-8% | 0-8% | 0-8% | 0-8% |
| B | 0-8% | 0-8% | 0-8% | 0-8% |
| Bi | 0-8% | 0-8% | 0-8% | 0-8% |
| Cr | 0-8% | 0-20% | 0-20% | 0-8% |
| Cu | 0-8% | 0-8% | 0-50% | 0-8% |
| Co | 0-8% | 0-8% | 0-8% | 0-8% |
| Fe | 0-8% | 0-8% | 0-8% | 0-8% |
| Hf | 0-8% | 0-8% | 0-8% | 0-8% |
| Ir | 0-8% | 0-8% | 0-8% | 0-12% |
| La | 0-8% | 0-8% | 0-8% | 0-8% |
| Mg | 0-8% | 0-8% | 0-8% | 0-8% |
| Mn | 0-8% | 0-8% | 0-8% | 0-8% |
| Mo | 0-55% | 40-93% | 0-50% | 0-20% |
| Nb | 0-8% | 0-8% | 0-8% | 40-85% |
| Ni | 0-45% | 0-8% | 0-8% | 0-8% |
| Os | 0-8% | 0-8% | 0-8% | 0-8% |
| Pt | 0-8% | 0-8% | 0-8% | 0-8% |
| Re | 14-40% | 7-40% | 7-40% | 7-40% |
| Rh | 0-8% | 0-8% | 0-8% | 0-8% |
| Se | 0-8% | 0-8% | 0-8% | 0-8% |
| Si | 0-8% | 0-8% | 0-8% | 0-8% |
| Sn | 0-8% | 0-8% | 0-8% | 0-8% |
| Ta | 35-84% | 0-50% | 0-50% | 0-35% |
| Tc | 0-8% | 0-8% | 0-8% | 0-8% |
| Ti | 0-8% | 0-8% | 0-8% | 0-8% |
| V | 0-8% | 0-8% | 0-8% | 0-8% |
| W | 0.1-25% | 0-50% | 14-10% | 0-15% |
| Y | 0-8% | 0-8% | 0-8% | 0-8% |
| Zr | 0-8% | 0-8% | 0-50% | 0-8% |

| Component/Wt. % | Ex. 81 | Ex. 82 | Ex. 83 | Ex. 84 |
|---|---|---|---|---|
| Ag | 0-5% | 0-5% | 0-5% | 0-5% |
| Al | 0-5% | 0-5% | 0-5% | 5-7% |
| B | 0-5% | 0-5% | 0-5% | 0-5% |
| Bi | 0-5% | 0-5% | 0-5% | 0-5% |
| Cr | 0-5% | 1-95% | 12-28% | 0-5% |
| Cu | 0-5% | 0-5% | 0-5% | 0-5% |
| Co | 0-5% | 0-5% | 36-68% | 0-5% |
| Fe | 0-5% | 0-5% | 0-18% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mr | 0-5% | 0-5% | 0-5% | 0-5% |
| Mo | 0-5% | 0-20% | 0-12% | 0-5% |
| Nb | 0-5% | 0-5% | 0-5% | 0-5% |
| Ni | 30-58% | 0-5% | 9-36% | 0-5% |
| Os | 0-5% | 0-5% | 0-5% | 0-5% |
| Pt | 0-5% | 0-5% | 0-5% | 0-5% |
| Re | 5-40% | 5-40% | 4.8-40% | 4.5-40% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 0-5% | 0-5% |
| Tc | 0-5% | 0-5% | 0-5% | 0-5% |
| Ti | 30-58% | 0-40% | 0-5% | 70-91.5% |
| V | 0-5% | 0-5% | 0-5% | 3-6% |
| W | 0-5% | 0-5% | 0-16% | 0-5% |
| Y | 0-5% | 0-5% | 0-5% | 0-5% |
| Zr | 0-5% | 0-20% | 0-5% | 0-5% |

| Component/Wt. % | Ex. 85 | Ex. 86 | Ex. 87 | Ex. 88 |
|---|---|---|---|---|
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 0-5% | 0-5% | 0-5% |
| Cu | 0-5% | 0-5% | 0-5% | 0-5% |
| Co | 0-5% | 0-5% | 0-5% | 0-5% |
| Fe | 0-5% | 0-5% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 1-15% | 2-10% | 3-8% | 0-5% |
| Nb | 0-5% | 0-5% | 0-5% | 20-45% |
| Ni | 0-5% | 0-5% | 0-5% | 0-5% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 0-5% | 1-15% |
| Tc | 0-5% | 0-5% | 0-5% | 0-5% |
| Ti | 51-70% | 51-70% | 55-70% | 51-70% |
| V | 0-5% | 0-5% | 0-5% | 0-5% |
| W | 0-5% | 0-5% | 0-5% | 0-5% |
| Y | 0-5% | 0-5% | 0-5% | 0-5% |
| Zr | 20-40% | 22-38% | 27-33% | 1-15% |
| Component/Wt. % | Ex. 89 | Ex. 90 | Ex. 91 | Ex. 92 |
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 0-5% | 0-5% | 0-5% |
| Cu | 0-5% | 0-5% | 0-5% | 0-5% |
| Co | 0-5% | 0-5% | 0-5% | 0-5% |
| Fe | 0-5% | 0-5% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-5% | 0-5% | 0-5% | 0-5% |
| Nb | 25-40% | 30-40% | 25-40% | 26-32% |
| Ni | 0-5% | 0-5% | 0-5% | 0-5% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 2-8% | 3-6% | 5-15% | 10-14% |
| Tc | 0-5% | 0-5% | 0-5% | 0-5% |
| Ti | 51-70% | 52-63% | 51-68% | 51-62% |
| V | 0-5% | 0-5% | 0-5% | 0-5% |
| W | 0-5% | 0-5% | 0-5% | 0-5% |
| Y | 0-5% | 0-5% | 0-5% | 0-5% |
| Zr | 2-12% | 4-8% | 2-8% | 2-6% |
| Component/Wt. % | Ex. 93 | Ex. 94 | Ex. 95 | Ex. 96 |
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 5-35% | 10-30% | 15-25% |
| Cu | 0-5% | 0-5% | 0-5% | 0-5% |
| Co | 0-5% | 20-55% | 25-50% | 35-45% |
| Fe | 0-5% | 3-25% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-5% | 2-15% | 3-12% | 4-9% |
| Nb | 30-40% | 0-5% | 0-5% | 0-5% |
| Ni | 0-5% | 4-23% | 5-20% | 10-18% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 1-3% | 0-5% | 0-5% | 0-5% |
| Tc | 0-5% | 0-5% | 0-5% | 0-5% |
| Ti | 51-67% | 0-5% | 0-5% | 0-5% |
| V | 0-5% | 0-5% | 0-5% | 0-5% |
| W | 0-5% | 0-5% | 0-5% | 0-5% |
| Y | 0-5% | 0-5% | 0-5% | 0-5% |
| Zr | 2-5% | 0-5% | 0-5% | 0-5% |

| Component/Wt. % | Ex. 97 | Ex. 98 | Ex. 99 | Ex. 100 |
|---|---|---|---|---|
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 0-5% | 0-5% | 0-5% |
| Cu | 0-5% | 0-5% | 0-5% | 0-5% |
| Co | 0-5% | 0-5% | 0-5% | 0-5% |
| Fe | 0-5% | 0-5% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 30-65% | 40-60% | 45-55% | 0-5% |
| Nb | 0-5% | 0-5% | 0-5% | 55-99.75% |
| Ni | 0-5% | 0-5% | 0-5% | 0-5% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 0-5% | 0-5% |
| Tc | 0-5% | 0-5% | 0-5% | 0-5% |
| Ti | 0-5% | 0-5% | 0-5% | 0-5% |
| V | 0-5% | 0-5% | 0-5% | 0-5% |
| W | 0-5% | 0-5% | 0-5% | 0-5% |
| Y | 0-5% | 0-5% | 0-5% | 0-5% |
| Zr | 30-56% | 40-60% | 45-55% | 0.25-45% |

| Component/Wt. % | Ex. 101 | Ex. 102 | Ex. 103 | Ex. 104 |
|---|---|---|---|---|
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 0-5% | 0-5% | 0-5% |
| Cu | 0-5% | 0-5% | 0-5% | 0-5% |
| Co | 0-5% | 0-5% | 0-5% | 0-5% |
| Fe | 0-5% | 0-5% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-5% | 0-5% | 0-5% | 0-5% |
| Nb | 75-99.5% | 95-99.25% | 55-78.5% | 68-74.25% |
| Ni | 0-5% | 0-5% | 0-5% | 0-5% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 20-35% | 25-30% |
| Tc | 0-5% | 0-5% | 0-5% | 0-5% |
| Ti | 0-5% | 0-5% | 0-5% | 0-5% |
| V | 0-5% | 0-5% | 0-5% | 0-5% |
| W | 0-5% | 0-5% | 1-8% | 0-5% |
| Y | 0-5% | 0-5% | 0-5% | 0-5% |
| Zr | 0.5-25% | 0.75-5% | 0.5-5% | 0.75-3% |

| Element/Wt. % | Ex. 105 | Ex. 106 | Ex. 107 | Ex. 108 |
|---|---|---|---|---|
| Re | 30-75% | 40-75% | 45-75% | 45-70% |
| Cr | 25-70% | 25-65% | 25-55% | 30-55% |
| Mo | 0-25% | 0-25% | 1-25% | 2-25% |
| Bi | 0-25% | 0-25% | 0-25% | 0-25% |
| Cr | 0-25% | 0-25% | 0-25% | 0-25% |
| Ir | 0-25% | 0-25% | 0-25% | 0-25% |
| Nb | 0-25% | 0-25% | 0-25% | 0-25% |
| Ta | 0-25% | 0-25% | 0-25% | 0-25% |
| V | 0-25% | 0-25% | 0-25% | 0-25% |
| W | 0-25% | 0-25% | 0-25% | 0-25% |
| Mn | 0-25% | 0-25% | 0-25% | 0-25% |
| Tc | 0-25% | 0-25% | 0-25% | 0-25% |
| Ru | 0-25% | 0-25% | 0-25% | 0-25% |
| Rh | 0-25% | 0-25% | 0-25% | 0-25% |
| Hf | 0-25% | 0-25% | 0-25% | 0-25% |
| Os | 0-25% | 0-25% | 0-25% | 0-25% |
| Cu | 0-25% | 0-25% | 0-25% | 0-25% |
| Ir | 0-25% | 0-25% | 0-25% | 0-25% |
| Ti | 0-25% | 0-25% | 0-25% | 0-25% |
| Y | 0-25% | 0-25% | 0-25% | 0-25% |
| Zr | 0-25% | 0-25% | 0-25% | 0-25% |

In Examples 1-108, it will be appreciated that all of the above ranges include any value between the range and any other range that is between the ranges set forth above. Any of the above values that include the ≤symbol includes the range from 0 to the stated value and all values and ranges therebetween.

In accordance with another and/or alternative aspect of the present disclosure, there is provided an expandable medical device medical device that is at least partially formed of a metal alloy and is configured to be radially collapsible to a collapsed or crimped state for introduction into at least a portion of a fractured bone (optionally via a sheath or guidewire) and radially expandable to an expanded state for implanting the expandable medical device at a fracture site of a fractured bone.

In accordance with another and/or alternative aspect of the present disclosure, there is provided an expandable medical device that can be optionally coated with a polymer material (e.g., silicone, PTFE, ePTFE, polyurethane, polyolefins, hydrogels, biological materials (e.g., pericardium or biological polymers such as collagen, gelatin, or hyaluronic acid derivatives), etc.). The coating can be used to partially or fully encapsulate the structures on the expandable medical device and/or to fill in openings on the expandable medical device.

In accordance with another and/or alternative aspect of the present disclosure, the metal alloy used to form at least a portion of the expandable medical device has one or more improved properties (e.g., strength, durability, hardness, biostability, bendability, coefficient of friction, radial strength, flexibility, tensile strength, tensile elongation, longitudinal lengthening, stress-strain properties, reduced recoil, radiopacity, heat sensitivity, biocompatibility, improved fatigue life, crack resistance, crack propagation resistance, reduced magnetic susceptibility, etc.), improved conformity when bent, less recoil, increased yield strength, improved fatigue ductility, improved durability, improved fatigue life, reduced adverse tissue reactions, reduced metal ion release, reduced corrosion, reduced allergic reaction, improved hydrophilicity, reduced toxicity, reduced thickness of metal component, improved bone fusion, and/or lower ion release into tissue. These one or more improved physical properties of the metal alloy can be achieved in the expandable medical device without having to increase the bulk and/or volume of the expandable medical device and, in some instances, these improved physical properties can be obtained even when the volume and/or bulk of the expandable medical device is reduced as compared to expandable medical devices at least partially formed from standard stainless steel, standard titanium alloy, or standard cobalt and chromium alloy materials. As such, the metal alloy used to at least partially form the expandable medical device can thus 1) increase the radiopacity of the expandable medical device, 2) increase the radial strength of the expandable medical device, 3) increase the yield strength and/or ultimate tensile strength of the expandable medical device, 4) improve the stress-strain properties of the expandable medical device, 5) improve the crimping and/or expansion properties of the expandable medical device, 6) improve the bendability and/or flexibility of the expandable medical device, 7) improve the strength and/or durability of the expandable medical device, 8) increase the hardness of the expandable medical device, 9) improve the recoil properties of the expandable medical device, 10) improve the biostability and/or biocompatibility properties of the expandable medical device, 11) increase fatigue resistance of the expandable medical device, 12) resist cracking in the expandable medical device and resist propagation of crack, 13) enable smaller, and/or thinner expandable medical devices to be made, 14) reduce the outer diameter of a crimped expandable medical device, 15) improve the conformity of the expandable medical device to the shape of the treatment area when the expandable medical device is used and/or expanded in the treatment area, 16) reduce the amount of recoil of the expandable medical device to the shape of the treatment area when the expandable medical device is expanded in the treatment area, 17) increase yield strength of the expandable medical device, 18) improve fatigue ductility of the expandable medical device, 18) improve durability of the expandable medical device, 19) improve fatigue life of the expandable medical device, 20) reduce adverse tissue reactions after implant of the expandable medical device, 21) reduce metal ion release after implant of the expandable medical device, 22) reduce corrosion of the expandable medical device after implant of the expandable medical device, 23) reduce allergic reaction after implant of the expandable medical device, 24) improve hydrophilicity of the expandable medical device, 25) reduce thickness of metal component of expandable medical device, 26) improve bone fusion with expandable medical device, 27) lower ion release from expandable medical device into tissue, 28) reduce magnetic susceptibility of the expandable medical device when implanted in a patient, and/or 29) reduce toxicity of the expandable medical device after implant of the expandable medical device.

In accordance with another and/or alternative aspect of the present disclosure, the expandable medical device is optionally subjected to one or more manufacturing processes. These manufacturing processes can include, but are not limited to, expansion, laser cutting, etching, crimping, annealing, drawing, pilgering, electroplating, electro-polishing, machining, plasma coating, 3D printed coatings, chemical vapor deposition, chemical polishing, cleaning, pickling, ion beam deposition or implantation, sputter coating, vacuum deposition, etc.

In accordance with another and/or alternative aspect of the present disclosure, the metal alloy optionally includes a certain amount of carbon and oxygen; however, this is not required. These two elements have been found to affect the forming properties and brittleness of the metal alloy. The controlled atomic ratio of carbon and oxygen of the metal alloy can also minimize the tendency of the metal alloy to form micro-cracks during the forming of the metal alloy into a frame for an expandable medical device, and/or during the use and/or expansion of the frame for an expandable medical device in a body. The carbon to oxygen atomic ratio can be as low as about 0.2:1 (e.g., 0.2:1 to 50:1 and all values and ranges therebetween). In one non-limiting formulation, the carbon to oxygen atomic ratio in the metal alloy is generally at least about 0.3:1. Typically the carbon content of the metal alloy is less than about 0.1 wt. % (e.g., 0-0.0999999 wt. % and all values and ranges therebetween), and more typically 0-0.01 wt. %. Carbon contents that are too large can adversely affect the physical properties of the metal alloy. Generally, the oxygen content is to be maintained at very low level. In one non-limiting formulation, the oxygen content is less than about 0.1 wt. % of the metal alloy (e.g., 0-0.0999999 wt. % and all values and ranges therebetween), and typically 0-0.01 wt. %.

In accordance with another and/or alternative aspect of the present disclosure, the metal alloy optionally includes a controlled amount of nitrogen; however, this is not required. Large amounts of nitrogen in the metal alloy can adversely affect the ductility of the metal alloy. This can in turn adversely affect the elongation properties of the metal alloy. In one non-limiting formulation, the metal alloy includes less than about 0.001 wt. % nitrogen (e.g., 0 wt. % to 0.0009999 wt. % and all values and ranges therebetween). It is believed that the nitrogen content should be less than the content of carbon or oxygen in the metal alloy. In one non-limiting formulation, the atomic ratio of carbon to nitrogen is at least about 1.5:1 (e.g., 1.5:1 to 400:1 and all values and ranges therebetween). In another non-limiting formulation, the atomic ratio of oxygen to nitrogen is at least about 1.2:1 (e.g., 1.2:1 to 150:1 and all value and ranges therebetween).

In accordance with another and/or alternative aspect of the present disclosure, the expandable medical device is generally designed to include at least about 5 wt. % of the metal alloy (e.g., 5-100 wt. % and all values and ranges therebetween).

In accordance with another and/or alternative aspect of the present disclosure, the metal alloy that is used to form all or part of the expandable medical device 1) is not clad, metal coated, metal sprayed, plated and/or formed (e.g., cold worked, hot worked, etc.) onto another metal, or 2) does not have another metal or metal alloy metal sprayed, coated, plated, clad and/or formed onto the metal alloy. It will be appreciated that in some applications, the metal alloy of the present disclosure may be clad, metal sprayed, coated, plated and/or formed onto another metal, or another metal or metal alloy may be plated, metal sprayed, coated, clad and/or formed onto the metal alloy when forming all or a portion of an expandable medical device.

In accordance with another and/or alternative aspect of the present disclosure, the metal alloy can be used to form a) a coating (e.g., cladding, dip coating, spray coating, plated coating, welded coating, plasma coating, etc.) on a portion of all of an expandable medical device, or b) a core of a portion or all of an expandable medical device. The composition of the coating is different from the composition of the material surface to which the metal alloy is coated. The coating thickness of the metal alloy is non-limiting (e.g., 1 μm to 1 inch and all values and ranges therebetween). In one non-limiting example, there is provided an expandable medical device wherein a core or base layer of the expandable medical device is formed of a metal or metal alloy (e.g., chromium alloy, titanium, titanium alloy, stainless steel, iron alloy, CoCr alloy, rhenium alloy, molybdenum alloy, tungsten alloy, Ta—W alloy, refractory metal alloy, MoTa alloy, MoRe alloy, etc.) or polymer or ceramic or composite material, and the other layer of the coated expandable medical device is formed of a different metal or metal alloy. The core or base layer and the other layer of the expandable medical device can each form 10-99% (and all values and ranges therebetween) of the overall cross section of the expandable medical device. When the outer metal coating is a rhenium containing alloy, such rhenium alloy can be used to create a hard surface on the expandable medical device at specific locations as well as all over the surface. In another non-limiting embodiment, the core or base layer of the expandable medical device can be formed of a rhenium containing alloy and the coating layer includes one or more other materials (e.g., another type of metal or metal alloy [e.g., chromium alloy, titanium, titanium alloy, stainless steel, iron alloy, CoCr alloy, rhenium alloy, molybdenum alloy, tungsten alloy, Ta—W alloy, refractory metal alloy, MoTa alloy, MoRe alloy, etc.), polymer coating, ceramic coating, composite material coating, etc.). Non-limiting benefits of using the rhenium containing alloy in the core or interior layer of the expandable medical device can include reducing the size of the expandable medical device, increasing the strength of the expandable medical device, and/or maintaining or reducing the cost of the expandable medical device. As can be appreciated, the use of the rhenium containing alloy can result in other or additional advantages. The core or base layer size and/or thickness of the metal alloy are non-limiting. In one non-limiting example, there is provided an expandable medical device that is at least partially formed from layered materials wherein a top layer is formed of material that is different form one or more other layers and the rhenium containing alloy forms one of the layers below the top layer, and the top layer is formed of a metal that is different from the rhenium containing alloy (e.g., chromium alloy, titanium, titanium alloy, stainless steel, iron alloy, CoCr alloy, rhenium alloy, molybdenum alloy, tungsten alloy, Ta—W alloy, refractory metal alloy, MoTa alloy, MoRe alloy, etc.). The core or lower layer or base layer and the outer layer of the layered material can each form 10-99% (and all values and ranges therebetween) of the overall cross section of the layered material.

In accordance with another and/or alternative aspect of the present disclosure, the expandable medical device can optionally be formed from into a shape that is at least 80% (e.g., 80-100% and all values and ranges therebetween) of the final net shape of the expandable medical device.

In another and/or alternative non-limiting embodiment of the disclosure, the average tensile elongation of the metal alloy used to at least partially form the expandable medical device is optionally at least about 20% (e.g., 20-50% average tensile elongation and all values and ranges therebetween). An average tensile elongation of at least 20% for the metal alloy is useful to facilitate in the expandable medical device being properly expanded when positioned in the treatment area of a body. The desired tensile elongation can be obtained from a unique combination of the metals in the metal alloy in combination with achieving the desired purity and composition of the alloy and the desired grain size of the metal alloy.

In accordance with another and/or alternative aspect of the present disclosure, the metal alloy is optionally at least partially formed by a swaging process; however, this is not required. In one non-limiting embodiment, swaging is performed on the metal alloy to at least partially or fully achieve final dimensions of one or more portions of the expandable medical device. The swaging dies can be shaped to fit the final dimension of the expandable medical device; however, this is not required.

In accordance with another and/or alternative aspect of the present disclosure, the metal alloy can optionally be nitrided; however, this is not required. The nitrided layer on the metal alloy can function as a lubricating surface during the optional drawing of the metal alloy when partially or fully forming the expandable medical device.

In accordance with another and/or alternative non-limiting aspect of the present disclosure, the expandable medical device can optionally be partially (e.g., 1% to 99.99% and all values and ranges therebetween) or fully be coated with and/or include one or more agents. The term “agent” includes, but is not limited to a substance, pharmaceutical, biologic, veterinary product, drug, and analogs or derivatives otherwise formulated and/or designed to prevent, inhibit and/or treat one or more clinical and/or biological events, and/or to promote healing. Non-limiting examples of clinical events that can be addressed by one or more agents include, but are not limited to, viral, fungus and/or bacterial infection; vascular diseases and/or disorders; lymphatic diseases and/or disorders; cancer; implant rejection; pain; nausea; swelling; organ failure; immunity diseases and/or disorders; cell growth inhibitors, blood diseases and/or disorders; heart diseases and/or disorders; neuralgia diseases and/or disorders; fatigue; genetic diseases and/or disorders; trauma; cramps; muscle spasms; tissue repair; nerve repair; neural regeneration and/or the like. The type and/or amount of agent included coated on expandable medical device can vary. In accordance with another and/or alternative aspect of the present disclosure, one or more portions of the expandable medical device can optionally 1) include the same or different agents, 2) include the same or different amount of one or more agents, 3) include the same or different polymer coatings, 4) include the same or different coating thicknesses of one or more polymer coatings, 5) have one or more portions of the expandable medical device controllably release and/or uncontrollably release one or more agents, and/or 6) have one or more portions of the expandable medical device controllably release one or more agents and one or more portions of the expandable medical device uncontrollably release one or more agents.

In accordance with another and/or alternative aspect of the present disclosure, one or more surfaces of the expandable medical device can optionally be treated to achieve the desired coating properties of the one or more agents and one or more polymers coated on the expandable medical device. Such surface treatment techniques include, but are not limited to, cleaning, buffing, smoothing, nitriding, annealing, swaging, cold working, etching (chemical etching, plasma etching, etc.), etc. As can be appreciated, other or additional surface treatment processes can be used prior to the coating of one or more agents and/or polymers on the surface of the expandable medical device.

In another and/or alternative non-limiting aspect of the disclosure, the expandable medical device can optionally include a marker material that facilitates enabling the expandable medical device to be properly positioned in a body passageway. The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.); and/or other types of electromagnetic waves (e.g., microwaves, visible light, infrared waves, ultraviolet waves, etc.).

In accordance with another and/or alternative aspect of the present disclosure, the expandable medical device or one or more regions of the expandable medical device can optionally be constructed by use of one or more microelectromechanical manufacturing (MEMS) techniques (e.g., micro-machining, laser micro-machining, micro-molding, etc.); however, other or additional manufacturing techniques can be used.

In accordance with another and/or alternative aspect of the present disclosure, the expandable medical device can optionally include one or more surface structures (e.g., pore, channel, pit, rib, slot, notch, bump, teeth, needle, well, hole, groove, etc.). These structures can be at least partially formed by MEMS (e.g., micro-machining, etc.) technology and/or other types of technology. In one non-limiting embodiment, the outer surface of at least a portion of the expandable medical device includes a plurality of ribs, bumps, teeth, and/or grooves that are used to engage an inner surface of intramedullary canal of the fractured bone so as to facilitate in anchoring at least a portion of the expandable medical device in the intramedullary canal when the expandable medical device is expanded in the intramedullary canal.

In accordance with another and/or alternative aspect of the present disclosure, the expandable medical device can optionally include one or more micro-structures (e.g., micro-needle, micro-pore, micro-cylinder, micro-cone, micro-pyramid, micro-tube, micro-parallelopiped, micro-prism, micro-hemisphere, teeth, rib, ridge, ratchet, hinge, zipper, zip-tie like structure, etc.) on the surface of the expandable medical device. As defined herein, a “micro-structure” is a structure having at least one dimension (e.g., average width, average diameter, average height, average length, average depth, etc.) that is no more than about 2 mm, and typically no more than about 1 mm.

In another and/or alternative aspect of the disclosure, the expandable medical device can optionally be an expandable device that can be expanded by use of some other device (e.g., balloon, etc.). The expandable medical device can be fabricated from a material that has no or substantially no shape-memory characteristics.

In accordance with another and/or alternative aspect of the present disclosure, there is optionally provided a near net process for the expandable medical device. In one non-limiting embodiment of the disclosure, there is provided a method of powder pressing materials and increasing the strength post-sintering by imparting additional cold work. In one non-limiting embodiment, the green part is pressed and then sintered. Thereafter, the sintered part is again pressed to increase its mechanical strength by imparting cold work into the pressed and sintered part.

In accordance with another and/or alternative aspect of the present disclosure, the metal alloy used to at least partially form the expandable medical device is initially formed into a blank, a rod, a tube, etc., and then finished into final form by one or more finishing processes. The metal alloy blank, rod, tube, etc., can be formed by various techniques such as, but not limited to, 1) melting the metal alloy and/or metals that form the metal alloy (e.g., vacuum arc melting, etc.) and then extruding and/or casting the metal alloy into a blank, rod, tube, etc., 2) melting the metal alloy and/or metals that form the metal alloy, forming a metal strip, and then rolling and welding the strip into a blank, rod, tube, etc., 3) consolidating the metal powder of the metal alloy and/or metal powder of metals that form the metal alloy into a blank, rod, tube, etc., or 4) 3-D printing the metal powder of the metal alloy and/or metal powder of metals that form the metal alloy into a blank, rod, tube, etc. When the metal alloy is formed into a blank, the shape and size of the blank is non-limiting.

In accordance with another and/or alternative aspect of the present disclosure, when the metal powder is consolidated to form the metal alloy into a blank, rod, tube, etc., the metal powder is pressed together to form a solid solution of the metal alloy into a near net expandable medical device, near net component of an expandable medical device, blank, rod, tube, etc. Typically, the pressing process is by an isostatic process (i.e., uniform pressure applied from all sides on the metal powder); however other processes can be used. When the metal powders are pressed together isostatically, cold isostatic pressing (CIP) is typically used to consolidate the metal powders; however, this is not required. The pressing process can be performed in an inert atmosphere, an oxygen-reducing atmosphere (e.g., hydrogen, argon and hydrogen mixture, etc.), and/or under a vacuum; however, this is not required.

In accordance with another and/or alternative aspect of the present disclosure, when the metal powder is used to 3D print an expandable medical device, component of an expandable medical device, blank, rod, tube, etc., the average particle size of the metal powder is optionally 2-62 microns, and more particularly about 5-49.9 microns, the average density of the metal powders is greater than 5 g/cm³, and the metal powder is generally spherical-shaped, and the Hall flow (s/50 g) is less than 30 seconds (e.g., 2-29.99 seconds and all values and ranges therebetween).

In accordance with another and/or alternative non-limiting aspect of the present disclosure, the expandable medical device can be partially (e.g., 1% to 99.99% and all values and ranges therebetween) or fully be coated with an enhancement coating to improve one or more properties of the expandable medical device (e.g., change exterior color of material having coated surface, increase surface hardness by use of the coated surface, increase surface toughness material having coated surface, reduced friction via use of the coated surface, improve scratch resistance of material that has the coated surface, improve impact wear of coated surface, improve resistance to corrosion and oxidation of coated material, form a non-stick coated surface, improve biocompatibility of material having the coated surface, reduce toxicity of material having the coated surface, reduce ion release from material having the coated surface, the enhancement coating forms a surface that is less of an irritant to cell about the coated surface after the expandable medical device is implanted, etc.). Non-limiting enhancement coatings that can be applied to a portion or all of the expandable medical device includes chromium nitride (CrN), diamond-like carbon (DLC), titanium nitride (TiN), titanium nitride oxide (TiNOx), zirconium nitride (ZrN), zirconium oxide (ZrO₂), zirconium-nitrogen-carbon (ZrNC), zirconium OxyCarbide (ZrOC), zirconium oxynitride (ZnNxOy) [e.g., cubic ZrN:O, cubic ZrO₂:N, tetragonal ZrO₂:N, and monoclinic ZrO₂:N phase coatings], and combinations of such coatings. In one non-limiting embodiment, the one or more enhancement coatings are optionally applied to a portion or all of the expandable medical device by a vacuum process using an energy source to vaporize material and deposit a thin layer of enhancement coating material. Such vacuum coating process, when used, can include a physical vapor deposition (PVD) process (e.g., sputter deposition, cathodic arc deposition or electron beam heating, etc.), chemical vapor deposition (CVD) process, atomic layer deposition (ALD) process, or a plasma-enhanced chemical vapor deposition (PE-CVD) process. In one non-limiting embodiment, the coating process is one or more of a PVD, CVD, ALD and PE-CVD, and wherein the coating process occurs at a temperature of 200-400° C. (and all values and ranges therebetween) for at least 10 minutes (e.g., 10-400 minutes and all values and ranges therebetween). In another non-limiting embodiment, the coating process is one or more of a PVD, CVD, ALD and PE-CVD, and wherein the coating process occurs at a temperature of 220-300° C. for 60-120 minutes. In another non-limiting embodiment, when the materials of the one or more enhancement coatings are to be applied to the outer surface of the expandable medical device that is partially or fully formed of a metal alloy, the materials of the one or more enhancement coatings can optionally be combine with one or more metals in the metal alloy, and/or combined with nitrogen, oxygen, carbon, or other elements that are in the metal alloy and/or present in the atmosphere about the metal alloy to a form an enhancement coating on the outer surface of the metal alloy. In another non-limiting embodiment, when the materials of the one or more enhancement coatings are to be applied to the outer surface of the expandable medical device that is partially or fully formed of a metal alloy, the materials of the one or more enhancement coatings can optionally be used to form various coating colors on the outer surface of the metal alloy (e.g., gold, copper, brass, black, rose gold, chrome, blue, silver, yellow, green, etc.). In another non-limiting embodiment, the thickness of the enhancement coating is greater than 1 nanometer (e.g., 2 nanometers to 100 microns and all values and ranges therebetween), and typically 0.1-25 microns, and more typically 0.2-10 microns. In another non-limiting embodiment, the hardness of the enhancement coating can be at least 5 GPa (ASTM C1327-15 or ASTM C1624-05), typically 5-50 GPa (and all values and ranges therebetween), more typically 10-25 GPa, and still more typically 14-24 GPa. In another non-limiting embodiment, the coefficient of friction (COF) of the enhancement coating can be 0.04-0.2 (and all values and ranges therebetween), and typically 0.6-0.15. In another non-limiting embodiment, the wear rate of the enhancement coating can be 0.5×10⁻⁷ mm³/N-m to 3×10⁻⁷ mm³/N-m (an all values and ranges therebetween), and typically 1.2×10⁻⁷ mm³/N-m to 2×10⁻⁷ mm³/N-m. In another non-limiting embodiment, silicon-based precursors (e.g., trimethysilane, tetramethylsilane, hexachlorodisilane, silane, dichlorosilane, trichlorosilane, silicon tetrachloride, tris(dimethylamino) silane, bis(tert-butylamino) silane, trisilylamine, allyltrimethoxysilanc, (3-aminopropyl)triethoxysilane, butyltrichlorosilane, n-sec-butyl (trimethylsilyl)amine, chloropentamethyldisilane, 1,2-dichlorotetramethyldisilane, [3-(diethylamino)propyl]trimethoxysilane, 1,3-diethyl-1,1,3,3-tetramethyldisilazane, dimethoxydimethylsilane, dodecamethylcyclohexasilane, hexamethyldisilane, isobutyl(trimethoxy)silane, methyltrichlorosilane, 2,4,6,8,10-pentamethylcyclopentasiloxane, pentamethyldisilane, n-propyltriethoxysilane, silicon tetrabromide, silicon tetrabromide, etc.) can optionally be used to facilitate in the application of the enhancement coating to one or more portions or all of the expandable medical device.

In accordance with another and/or alternative non-limiting aspect of the present disclosure, the expandable medical device can be partially or fully coated with an enhancement coating composition that includes a chromium nitride (CrN) coating. A portion or all of the expandable medical device can be partially or fully coated with the chromium nitride (CrN) coating. The enhancement coating can be used to improve hardness, improve toughness, reduced friction, resistant impact wear, improve resistance to corrosion and oxidation, and/or form a reduced stick surface when in contact with many different materials. In accordance with one non-limiting embodiment, the chromium nitride (CrN) coating generally includes 40-85 wt. % Cr (and all values and ranges therebetween), 15-60 wt. % N (and all values and ranges therebetween), 0-10 wt. % Re (and all values and ranges therebetween), 0-10 wt. % Si (and all values and ranges therebetween), 0-2 wt. % O (and all values and ranges therebetween), and 0-2 wt. % C (and all values and ranges therebetween). In one non-limiting coating process, all or a portion of the expandable medical device are initially coated with Cr metal. The Cr metal coating can be applied by PVD, CVD, ALD and PE-CVD in an inert environment. The coating thickness of Cr metal is 0.5-15 microns. Thereafter, the Cr metal coating is exposed to nitrogen gas and/or a nitrogen containing gas compound to cause the nitrogen to react with the Cr metal coating to form a layer of CrN on the outer surface of the Cr metal coating and/or the outer surface of the expandable medical device. Particles of Cr metal can optionally be mixed with nitrogen gas and/or a nitrogen containing gas compound to facilitate in the formation of the CrN coating. When Cr metal particles are used, the initial Cr coating layer on the expandable medical device can optionally be eliminated. In another non-limiting embodiment, the enhancement coating composition generally includes 65-80 wt. % Cr, 15-30 wt. % N, 0-8 wt. % Re, 0-1 wt. % Si, 0-1 wt. % O, and 0-1 wt. % C.

In accordance with another and/or alternative non-limiting aspect of the present disclosure, the expandable medical device can be partially or fully coated with an enhancement coating composition that includes a diamond-Like Carbon (DLC) coating. A portion or all of the expandable medical device can be partially or fully coated with the diamond-Like Carbon (DLC) coating. The enhancement coating can be used to improve hardness, improve toughness, reduced friction, resistant impact wear, improve resistance to corrosion and oxidation, improve biocompatibility, and/or form a reduced stick surface when in contact with many different materials. In one non-limiting embodiment, the diamond-Like Carbon (DLC) coating generally includes 60-99.99 wt. % C (and all values and ranges therebetween), 0-2 wt. % N (and all values and ranges therebetween), 0-10 wt. % Re (and all values and ranges therebetween), 0-20 wt. % Si (and all values and ranges therebetween), and 0-2 wt. % O (and all values and ranges therebetween). The carbon coating can be applied by PVD, CVD, ALD and PE-CVD in an inert environment. The carbon layer can be applied by use of methane and/or acetylene gas; however, other or additional carbon sources can be used. The coating thickness of the carbon is 0.5-15 microns. In another non-limiting embodiment, all or a portion of the expandable medical device are coated with the enhancement coating composition that generally includes 90-99.99 wt. % C, 0-1 wt. % N, 0-8 wt. % Re, 0-1 wt. % Si, and 0-1 wt. % O.

In accordance with another and/or alternative non-limiting aspect of the present disclosure, the expandable medical device can be partially or fully coated with an enhancement coating composition that includes a titanium nitride (TiN) coating. A portion or all of the outer surface of the expandable medical device can include the titanium nitride (TiN) coating. The enhancement coating can be used to improve hardness, improve toughness, improve resistance to corrosion and oxidation, reduced friction, and/or form a reduced stick surface when in contact with many different materials. In one non-limiting embodiment, all or a portion of the outer surface of the expandable medical device are optionally initially coated with Ti metal. The Ti metal coating, when applied, can be applied by PVD, CVD, ALD and PE-CVD in an inert environment. The coating thickness of Ti metal is 0.05-15 microns (and all values and ranges therebetween). As can be appreciated, the initial Ti coating is optional. Thereafter, the Ti metal coating, when applied, is exposed to nitrogen gas and/or a nitrogen containing gas compound and optionally titanium particles to cause the nitrogen to react with the Ti metal coating and/or titanium metal particles to form a layer of TiN on the outer surface of the Ti metal coating and/or the outer surface of the expandable medical device. If a titanium layer is not preapplied, the TiN coating can be formed by exposing the expandable medical device to titanium particles and nitrogen gas and/or a nitrogen containing gas compound. The coating thickness of the TiN coating is generally 0.1-15 microns (and all values and ranges therebetween), and typically 0.2-2 microns.

In accordance with another and/or alternative non-limiting aspect of the present disclosure, the expandable medical device can be partially or fully coated with an enhancement coating composition that includes a titanium nitride oxide (TiNOx) coating. A portion or all of the outer surface of the expandable medical device can include the titanium nitride oxide (TiNOx) coating. The enhancement coating can be used to improve hardness, improve toughness, improve resistance to corrosion and oxidation, reduced friction, and/or form a reduced stick surface when in contact with many different materials. In one non-limiting embodiment, all or a portion of the outer surface of the expandable medical device are optionally initially coated with Ti metal. The Ti metal coating, when applied, can be applied by PVD, CVD, ALD and PE-CVD in an inert environment. The coating thickness of Ti metal is 0.05-15 microns (and all values and ranges therebetween). As can be appreciated, the initial Ti coating is optional. Thereafter, the Ti metal coating is exposed to titanium particles and a nitrogen and oxygen mixture that can include nitrogen gas, oxygen gas, a nitrogen containing gas compound and/or an oxygen containing gas compound to cause the nitrogen and oxygen to react with the Ti metal coating, if such coating is used, and/or with the Ti metal particles to form a layer of TiNOx on the outer surface of the Ti metal coating and/or the outer surface of the expandable medical device. The ratio of the N to the O can be varied to control the about of O in the TiNOx coating. If a titanium layer is not preapplied, the TiNOx coating can be formed by exposing the expandable medical device to titanium particles and a nitrogen and oxygen source such as nitrogen gas, oxygen gas, a nitrogen containing gas compound and/or an oxygen containing gas compound. The ratio of N to O when forming the TiNOx coating is generally 1:10 to 10:1 (and all values and ranges therebetween). The coating thickness of the TiNOx coating is generally 0.1-15 microns (and all values and ranges therebetween), and typically 0.2-2 microns. In another non-limiting embodiment, a TiNOx coating is applied to a portion or all of the outer surface of the expandable medical device, and the TiNOx coating is formed by a) exposing the outer surface of a portion of all of the expandable medical device to Ti particles (PVD, CVD, ALD and PE-CVD process) and/or a Ti containing solution to form a Ti layer on a portion of all of the expandable medical device, and wherein the thickness of the Ti coating is 0.05-5 microns, and b) exposing the Ti coating to a nitrogen and oxygen source such as nitrogen gas, oxygen gas, a nitrogen containing gas compound and/or an oxygen containing gas compound to form a TiNOx coating, and wherein ratio of N to O when forming the TiNOx coating is generally 1:10 to 10:1, and wherein the coating thickness of the TiNOx coating is 0.2-5 microns. In another non-limiting embodiment, a TiNOx coating is applied to a portion or all of the outer surface of the expandable medical device, and the TiNOx coating is formed by exposing a portion or all of the outer surface of the expandable medical device to Ti particles and a nitrogen and oxygen source such as nitrogen gas, oxygen gas, a nitrogen containing gas compound and/or an oxygen containing gas compound to form a TiNOx coating, and wherein ratio of N to O when forming the TiNOx coating is generally 1:10 to 10:1, and wherein the coating thickness of the TiNOx coating is 0.2-5 microns. In another non-limiting embodiment, the enhancement coating composition generally includes 20-85 wt. % Ti (and all values and ranges therebetween), 0.5-35 wt. % N (and all values and ranges therebetween), 0-10 wt. % Re (and all values and ranges therebetween), and 0.5-35 wt. % O (and all values and ranges therebetween). In another non-limiting embodiment, a coating of TiNOx was formed on the expandable medical device by reactive physical vapor deposition in a vacuum chamber. Depending on the oxygen-nitrogen ratio during vapor deposition, a coating deposit of TiNOx with defined composition and resistivity can be coated on the outer surface of the expandable medical device.

In accordance with another and/or alternative non-limiting aspect of the present disclosure, the expandable medical device can be partially or fully coated with an enhancement coating composition that includes a zirconium nitride (ZrN) coating. The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the expandable medical device is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to nitrogen gas and/or a nitrogen containing gas compound to cause the nitrogen to react with the Zn metal coating to form a layer of ZIN on the outer surface of the Zr metal coating and/or the outer surface of the expandable medical device. Particles of Zr metal can optionally be mixed with nitrogen gas and/or a nitrogen containing gas compound to facilitate in the formation of the ZrN coating. When Zr metal particles are used, the initial Zr coating layer on the expandable medical device can optionally be eliminated. The ZrN coating has been found to produce a gold colored enhancement coating color. In another non-limiting embodiment, the enhancement coating composition generally includes 35-90 wt. % Zr (and all values and ranges therebetween), 5-25 wt. % N (and all values and ranges therebetween), 0-10 wt. % Re (and all values and ranges therebetween), 0-20 wt. % Si (and all values and ranges therebetween), 0-2 wt. % O (and all values and ranges therebetween), and 0-2 wt. % C (and all values and ranges therebetween). In another non-limiting embodiment, the enhancement coating composition generally includes 80-90 wt. % Zr, 10-20 wt. % N, 0-8 wt. % Re, 0-1 wt. % Si, 0-1 wt. % O, and 0-1 wt. % C.

In accordance with another and/or alternative non-limiting aspect of the present disclosure, the expandable medical device can be partially or fully coated with an enhancement coating composition that includes a zirconium oxide (ZrO₂) coating. The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the expandable medical device is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to oxygen gas and/or oxygen containing gas compound to cause the oxygen to react with the Zn metal coating to form a layer of zirconium oxide (ZrO₂) on the outer surface of the Zr metal coating and/or the outer surface of the expandable medical device. Particles of Zr metal can optionally be mixed with the oxygen gas and/or an oxygen containing gas compound to facilitate in the formation of the ZrO₂ coating. When Zr metal particles are used, the initial Zr coating layer on the expandable medical device can optionally be eliminated. The zirconium oxide (ZrO₂) coating has been found to produce a blue colored enhancement coating color. In another non-limiting embodiment, the enhancement coating composition generally includes 35-90 wt. % Zr (and all values and ranges therebetween), 10-35 wt. % O (and all values and ranges therebetween), 0-2 wt. % N (and all values and ranges therebetween), 0-10 wt. % Re (and all values and ranges therebetween), 0-20 wt. % Si (and all values and ranges therebetween), and 0-2 wt. % C (and all values and ranges therebetween). In another non-limiting embodiment, the enhancement coating composition generally includes 70-80 wt. % Zr, 20-30 wt. %, 0-1 wt. % N, 0-8 wt. % Re, 0-1 wt. % Si, and 0-1 wt. % C.

In accordance with another and/or alternative non-limiting aspect of the present disclosure, the expandable medical device can be partially or fully coated with an enhancement coating composition that includes both a zirconium oxide (ZrO₂) coating and a zirconium nitride coating (ZrN). The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the metal alloy is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to a) both oxygen gas and/or oxygen containing gas compound and also to nitrogen gas and/or nitrogen containing gas compound, b) nitrogen gas and/or nitrogen containing gas compound and then to oxygen gas and/or oxygen containing gas compound, or c) oxygen gas and/or oxygen gas containing compound and then to nitrogen gas and/or nitrogen gas containing compound. The coating composition of the zirconium oxide (ZrO₂) coating and the zirconium nitride coating (ZrN) are similar or the same as discussed above. As discussed above, Particles of Zr metal can optionally be mixed with the oxygen gas and/or an oxygen containing gas compound to facilitate in the formation of the ZrO₂ coating and the nitrogen gas and/or nitrogen gas containing compound to facilitate in the formation of the ZrN coating. When Zr metal particles are used, the initial Zr coating layer on the expandable medical device can optionally be eliminated.

In accordance with another and/or alternative non-limiting aspect of the present disclosure, the expandable medical device can be partially or fully coated with an enhancement coating composition that includes a zirconium oxycarbide (ZrOC) coating. The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the metal alloy is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to a) both to oxygen gas and/or an oxygen containing gas compound and to carbon and/or a carbon containing gas compound (e.g., methane and/or acetylene gas), b) carbon and/or a carbon containing gas compound and then to oxygen gas and/or an oxygen containing gas compound, or c) oxygen gas and/or oxygen containing gas compound and then to carbon and/or carbon containing gas compound. Particles of Zr metal can optionally be mixed with the oxygen gas and/or an oxygen containing gas compound and the carbon and/or carbon containing gas compound to facilitate in the formation of the zirconium oxycarbide (ZrOC) coating. When Zr metal particles are used, the initial Zr coating layer on the expandable medical device can optionally be eliminated. In another non-limiting embodiment, the enhancement coating composition generally includes 40-95 wt. % Zr (and all values and ranges therebetween), 5-25 wt. % O (and all values and ranges therebetween), and 10-40 wt. % C (and all values and ranges therebetween), 0-2 wt. % N (and all values and ranges therebetween), 0-10 wt. % Re (and all values and ranges therebetween), and 0-20 wt. % Si (and all values and ranges therebetween). In another non-limiting embodiment, the enhancement coating composition generally includes 40-65 wt. % Zr, 5-25 wt. % O, and 25-40 wt. % C, 0-1 wt. % N, 0-8 wt. % Re, and 0-1 wt. % Si.

In accordance with another and/or alternative non-limiting aspect of the present disclosure, one or more components of the expandable medical device can be partially or fully coated with an enhancement coating composition that includes a zirconium oxynitride (ZnNxOy) [e.g., cubic ZrN:O, cubic ZrO₂:N, tetragonal ZrO₂:N, and monoclinic ZrO₂:N phase coatings]. A portion or all of the outer surface of the one or more components of the expandable medical device can include the zirconium oxynitride (ZnNxOy). The enhancement coating can be used to improve hardness, improve toughness, improve resistance to corrosion and oxidation, reduced friction, form a reduced stick surface when in contact with many different materials, and/or promote nitric oxide formation on the surface of the coating. In one non-limiting embodiment, all or a portion of the outer surface of the one or more components of the expandable medical device are optionally initially coated with Zr metal. The Zr metal coating, when applied, can be applied by PVD, CVD, ALD and PE-CVD in an inert environment. The coating thickness of Zr metal is 0.05-15 microns (and all values and ranges therebetween). As can be appreciated, the initial Zr coating is optional. Thereafter, the Zr metal coating is exposed to zirconium particles and a nitrogen and oxygen mixture that can include nitrogen gas, oxygen gas, a nitrogen containing gas compound and/or an oxygen containing gas compound to cause nitrogen and oxygen to react with the Zr metal coating, if such coating is used, and/or with the Zr metal particles to form a layer of ZnNxOy on the outer surface of the Zr metal coating and/or the outer surface of the one or more components of the expandable medical device. The ratio of the N to the O can be varied to control the about of O and N in the ZrNxOy coating. If a zirconium layer is not preapplied, the ZrNxOy coating can be formed by exposing the outer surface of one or more components of the expandable medical device to zirconium particles and a nitrogen and oxygen source such as nitrogen gas, oxygen gas, a nitrogen containing gas compound and/or an oxygen containing gas compound. The ratio of N to O when forming the ZrNxOy coating is generally 1:10 to 10:1 (and all values and ranges therebetween). The coating thickness of the ZrNxOy coating is generally 0.1-15 microns (and all values and ranges therebetween), and typically 0.2-2 microns. In another non-limiting embodiment, a ZrNxOy coating is applied to a portion or all of the outer surface of the one or more components of the expandable medical device, and the ZrNxOy coating is formed by a) exposing the outer surface of a portion of all of the one or more components of the expandable medical device to Zr particles (PVD, CVD, ALD and PE-CVD process) and/or a Zr containing solution to form a Zr layer on a portion of all of the one or more components of the expandable medical device, and wherein the thickness of the Zr coating is 0.05-5 microns, and b) exposing the Zr coating to a nitrogen and oxygen source such as nitrogen gas, oxygen gas, a nitrogen containing gas compound and/or an oxygen containing gas compound to form a ZrNxOy coating, and wherein ratio of N to O when forming the ZrNxOy coating is generally 1:10 to 10:1, and wherein the coating thickness of the ZrNxOy coating is 0.2-5 microns. In another non-limiting embodiment, a ZrNxOy coating is applied to a portion or all of the outer surface of the one or more components of the expandable medical device, and the ZrNxOy coating is formed by exposing a portion or all of the outer surface of the one or more components of the expandable medical device to Zr particles and a nitrogen and oxygen source such as nitrogen gas, oxygen gas, a nitrogen containing gas compound and/or an oxygen containing gas compound to form a ZrNxOy coating, and wherein ratio of N to O when forming the ZrNxOy coating is generally 1:10 to 10:1, and wherein the coating thickness of the ZrNxOy coating is 0.2-5 microns. In another non-limiting embodiment, the enhancement coating composition generally includes 20-85 wt. % Zr (and all values and ranges therebetween), 0.5-35 wt. % N (and all values and ranges therebetween), and 0.5-35 wt. % O (and all values and ranges therebetween). In another non-limiting embodiment, a coating of ZrNxOy was formed on one or more components of the expandable medical device by reactive physical vapor deposition in a vacuum chamber. Depending on the oxygen-nitrogen ratio during vapor deposition, a coating deposit of ZrNxOy with defined composition and resistivity can be coated on the outer surface of the one or more components of the expandable medical device.

In accordance with another and/or alternative non-limiting aspect of the present disclosure, the expandable medical device can be partially or fully coated with an enhancement coating composition that includes a zirconium-nitrogen-carbon (ZrNC) coating. The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the expandable medical device is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to nitrogen gas and/or a nitrogen containing gas compound and then to carbon and/or a carbon containing gas compound (e.g., methane and/or acetylene gas). The color of the ZrNC will vary depending on the amount of C and N in the coating. Particles of Zr metal can optionally be mixed with nitrogen gas and/or a nitrogen containing gas compound and the carbon and/or a carbon containing gas compound to facilitate in the formation of the ZrNC coating. When Zr metal particles are used, the initial Zr coating layer on the expandable medical device can optionally be eliminated. In one non-limiting embodiment, the enhancement coating composition generally includes 40-95 wt. % Zr (and all values and ranges therebetween), 5-40 wt. % N (and all values and ranges therebetween), and 5-40 wt. % C (and all values and ranges therebetween), 0-2 wt. % O (and all values and ranges therebetween), 0-10 wt. % Re (and all values and ranges therebetween), and 0-20 wt. % Si (and all values and ranges therebetween). In another non-limiting embodiment, the enhancement coating composition generally includes 40-80 wt. % Zr, 5-25 wt. % N, and 5-25 wt. % C, 0-1 wt. % O, 0-8 wt. % Re, and 0-1 wt. % Si.

In accordance with another and/or alternative non-limiting aspect of the present disclosure, the expandable medical device is configured to be at least partially or fully positioned in the intramedullary canal of a bone. The expandable medical device is configured to be radially collapsible to a collapsed or crimped state for introduction into the intramedullary canal of a bone and radially expandable to an expanded state for implanting the expandable medical device at a desired location in the intramedullary canal of the bone. The expandable medical device is formed of a plastically-expandable material that permits crimping of the expandable medical device to a smaller profile for delivery and expansion of the expandable medical device once positioned in the desired location in the intramedullary canal of a bone. The expansion of the crimped frame of the expandable medical device can be by an expansion device such as, but not limited to, a balloon of on a balloon catheter. During the insertion of the expandable medical device into the intramedullary canal of a bone, the expandable medical device can optionally be at least partially surrounded by a flexible sheath. During the insertion of the expandable medical device into the intramedullary canal of a bone, the expandable medical device can optionally be at least partially guided into the intramedullary canal by a guidewire. Prior to, during and/or after the expandable medical device is at least partially positioned in the intramedullary canal of a bone, a portion or all of the intramedullary canal of a bone that includes the expandable medical device can optionally be filled with a column of surgical fluid (e.g., polymer cement, resin, etc.). The expandable medical device is configured to span a fracture site in a bone. The expandable medical device can optionally be introduced into the intramedullary canal of a bone through an incision in the skin and an opening or breach in the fractured bone, along a path which can be along a longitudinal axis of the canal in the bone. Once the expandable medical device is properly positioned in the bone, a cement, adhesive or other surgical fluid can optionally be used to provide fixation and stabilization of the expandable medical device at the bone fracture site. The expandable medical device can be used for fixation and stabilization of long bone fractures; however, the expandable medical device can be used for fixation and stabilization other bones and structures as well. In another non-limiting embodiment, the expandable medical device can be configured to press against or otherwise engage the interior endosteal surface of the bone. The expandable medical device, during its placement and expansion, can be used to assist in moving and/or aligning one or more bone fragments. Re-integration of bone fragments by the expandable medical device can be used to facilitate in the fracture healing process. The expandable medical device can optionally be used to a) aid in the reduction of the fracture by exerting outward forces and pressure from within the intramedullary canal of the fractured bone, and/or b) facilitate the movement or alignment of one or more bone fragments of the fractured bone toward or to a more beneficial or desired location.

In accordance with another and/or alternative non-limiting aspect of the present disclosure, the expandable medical device can be sized and shaped to a) expand with sufficient force to facilitate the movement and re-positioning of one or more bone fragments of a fractured bone that have crushed into or otherwise encroached upon the intramedullary canal (e.g., used to drive such bone fragments radially outward and generally toward a position more aligned with the opposing fracture ends, thereby promoting re-integration of the fragments during the fracture healing process), b) expand to fill gaps or voids in the cortical bone wall or endosteum at or near the fracture site (e.g., if one or more bone fragments have separated away from the fracture site and are not compressed, aligned, or otherwise brought back nearer the fracture site, the expandable medical device can be configured to expand to fill the space once occupied by an absent fragment, and/or c) provide support to the fracture site without requiring additional structures (e.g., sheath, surgical fluid, etc.).

In accordance with another and/or alternative non-limiting aspect of the present disclosure, the expandable medical device can optionally include one or more gripping members disposed on or along the outer surface of the expandable medical device to provide a secure attachment to the inside walls of the intramedullary canal of the bone.

In accordance with another and/or alternative non-limiting aspect of the present disclosure, the expandable medical device has one or more of the following advantages: a) less invasive (e.g., upper extremity 1-3 mm puncture; lower extremity 2-6 mm puncture), b) no screws or plates impinging tendons, muscles etc., c) full internal fixation within the bone, d) preserves bone marrow which is essential to healing, e) insertion sheath for the expandable medical device allows ready outflow for bone marrow getting pushed out and could be reinjected into the bone after insertion of the expandable medical device in the bone, f) reduces risk of pulmonary embolism from bone marrow embolization, g) the expandable medical device can be designed to foreshorten so as to create a built in mechanism for fracture reduction/compression during the expansion of the expandable medical device, h) ends of the expandable medical device can be first expanded to cause fixation of the ends of the expandable medical device in the bone prior to full expansion of the middle portion of the expandable medical device, which expansion of the middle portion of the expandable medical device creates longitudinal compression of the fractured bone due to the foreshortening of the expandable medical device during expansion of the middle portion of the expandable medical device, i) no need for screws to anchor the ends of the expandable medical device in the bone during the expansion of the expandable medical device in the bone (e.g., typical balloon pressures are 10-20 atmospheres ˜150 to 300 pounds per square inch when expanding expandable medical device, thus such expansion forces should secure the ends of the expandable medical device within the bone without needs of screws; however, screws can still be used if needed, and/or j) the expandable medical device can include one or more markers to facilitate in the placement of the expandable medical device in the fractured bone.

In accordance with another and/or alternative non-limiting aspect of the present disclosure, a second expandable medical device can be optionally inserted in the interior of a first expanded expandable medical device to increase the strength and/or rigidity of the expandable medical device system at the bone fracture. For example, a first expandable medical device can be inserted and expanded in the bone fracture. Thereafter, a second expandable medical device can optionally be partially or fully inserted into the interior of a portion or all of the expanded first expandable medical device. Generally, the longitudinal length of the expanded second expandable medical device is less (e.g., 20-80% less and all values and ranges therebetween) than the longitudinal length of the expanded first expandable medical device; however, this is not required. After the second expandable medical device is partially or fully inserted into the interior of a portion or all of the expanded first expandable medical device, and thereafter expended, the expanded second expandable medical device generally expands the length of the bone fracture. The second expandable medical device can be configured to a) foreshorten when expanded such that the longitudinal length of the expanded second expandable medical device is at least 15% less (e.g., 15-60% less and all values and ranges therebetween) than a longitudinal length of the crimped or non-expanded second expandable medical device, or b) not or substantially not foreshorten when expanded such that the longitudinal length of the expanded second expandable medical device is no more than 10% less (e.g., 0-10% less and all values and ranges therebetween) than a longitudinal length of the crimped or non-expanded second expandable medical device. When the expanded second expandable medical device is configured to foreshorten, the reduced longitudinal length of the expanded second expandable medical device can facilitate in further drawing the fractured bones together. The use of the expanded second expandable medical device can result in increased rigidity and/or strength in the region of the two expanded expandable medical devices so as to provide additional supported to the fracture region of the bone. The material used to form the first and second expandable medical devices can be the same or different. One or both of the first and second expandable medical devices can include an enhancement coating and/or biological agent.

One non-limiting object of the present disclosure is the provision of an expandable medical device that can be partially or fully inserted into a fractured bone to facilitate in the fixation, repair and stabilization of the fractured bone

In another and/or alternative non-limiting object of the present disclosure is the provision of an expandable medical device that is partially or fully formed of a rhenium containing metal alloy that can be partially or fully inserted into a fractured bone to facilitate in the fixation, repair and stabilization of the fractured bone.

In another and/or alternative non-limiting object of the present disclosure is the provision of an expandable medical device that is partially or fully formed of a rhenium containing metal alloy that can be partially or fully inserted into a fractured bone to facilitate in the fixation, repair and stabilization of the fractured bone, and wherein the metal alloy used to partially or fully form the expandable medical device has a sufficient quantity of rhenium such that the ductility and tensile strength of the metal alloy is improved as compared to a similar metal alloys that are absent rhenium.

In another and/or alternative non-limiting object of the present disclosure is the provision of an expandable medical device that is partially or fully formed of a rhenium containing metal alloy that exhibits a rhenium effect.

In another and/or alternative non-limiting object of the present disclosure is the provision of an expandable medical device that is partially or fully formed of a rhenium containing metal alloy that exhibits a rhenium effect and includes at least 15 awt. % rhenium.

In another and/or alternative non-limiting object of the present disclosure is the provision of using a second expandable medical device that is inserted into the interior of a first expanded expandable medical device to increase the strength and/or rigidity of the expandable medical device system at the bone fracture.

In another and/or alternative non-limiting object of the present disclosure is the provision of an expandable medical device for treating a fracture site in a fractured bone having an intramedullary canal; the expandable medical device includes an expandable frame with an open cell configuration; the expandable frame includes a plurality of interconnected struts; the expandable frame has an unexpanded shape and size that enables the expandable frame to be insert into the intramedullary canal; the expandable frame has an expanded shape and size that enables the expandable frame to be secured in the intramedullary canal while traversing a fracture site of the fractured bone; the expandable frame has a longitudinal length that is sufficient to fully span the fracture site; the expandable frame is expandable from a first cross-sectional size in the unexpanded state to a second cross-sectional size in the expanded state; a cross-sectional area of the expandable frame in the second cross-sectional size is larger than a cross-sectional area of the expandable frame in the first cross-sectional size; the longitudinal length of the expandable frame in the unexpanded state is greater than the longitudinal length of the expandable frame in the expanded state; the expandable frame has a side wall that includes a plurality of openings; the expandable frame is at least partially formed of metal alloy that includes at least 5 awt. % (e.g., 5-99 awt. % and all values and ranges therebetween) rhenium and additive material; the additive material includes one or more metals that are selected from the group consisting of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and zirconium; the rhenium and the additive material constitutes at least 90 wt. % of the rhenium alloy.

In another and/or alternative non-limiting object of the present disclosure is the provision of an expandable medical device that is at least partially coated with a biocompatible material; the biocompatible material includes a) biological agent, b) titanium nitride oxide (TiNOx) coating, c) titanium nitride (TiN) coating, d) chromium nitride (CrN) coating, e) diamond-like carbon (DLC) coating, f) zirconium nitride (ZrN) coating, g) zirconium oxide (ZrO2) coating, h) zirconium-nitrogen-carbon (ZrNC) coating, i) zirconium OxyCarbide (ZrOC) coating, and/or j) zirconium oxynitride (ZrNxOy) coating.

In another and/or alternative non-limiting object of the present disclosure is the provision of a method for repairing a bone that is fractured comprising: a) providing a fractured bone that includes first and second bone portions and a fracture site that is located between the first and second bone portions; the fracture site has a fracture site width; each of the first and second bone portions of the fractured bone includes an intramedullary canal; b) providing an expandable medical device for treating the fracture site in the fractured bone; the expandable medical device includes an expandable frame with an open cell configuration; the expandable frame includes a plurality of interconnected struts; the expandable frame has an unexpanded shape and size that enables the expandable frame to be insert into the intramedullary canal; the expandable frame has an expanded shape and size that enables the expandable frame to be secured in the intramedullary canal while traversing a fracture site of the fractured bone; the expandable frame has a longitudinal length that is sufficient to fully span the fracture site; the expandable frame is expandable from a first cross-sectional size in the unexpanded state to a second cross-sectional size in the expanded state; a cross-sectional area of the expandable frame in the second cross-sectional size is larger than a cross-sectional area of the expandable frame in the first cross-sectional size; the longitudinal length of the expandable frame in the unexpanded state is greater than the longitudinal length of the expandable frame in the expanded state; the expandable frame has a side wall that includes a plurality of openings; the expandable frame is at least partially formed of metal alloy that includes at least 5 awt. % (e.g., 5-99 awt. % and all values and ranges therebetween) rhenium and additive material; the additive material includes one or more metals that are selected from the group consisting of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and zirconium; the rhenium and the additive material constitutes at least 90 wt. % of the rhenium alloy; c) inserting the expandable medical device in the intramedullary canal while the expandable medical device in the unexpanded state such that at least a portion of the expandable medical device is positioned in the first and second bone portions and traverses the fracture site; and d) expanding the expandable medical device in the intramedullary canal to the expanded state to repair the fractured bone; and wherein expansion of the expandable medical device causes the first and second bone portions to thereby cause a reduction in the fracture site width.

In another and/or alternative non-limiting object of the present disclosure is the provision of a method for repairing a bone that is fractured wherein the expandable medical device includes a proximal portion, a distal portion and a mid-portion; and wherein the step of expanding includes expanding the proximal portion and/or a distal portion of the expandable medical device prior to expanding the mid-portion; and wherein prior expansion of the proximal portion and/or a distal portion causes the proximal portion and/or a distal portion to be at least partially anchored in the intramedullary canal prior to expansion of the mid-portion.

In another and/or alternative non-limiting object of the present disclosure is the provision of a method for repairing a bone that is fractured wherein the expandable medical device includes a proximal portion, a distal portion and a mid-portion; and further including the step of securing the proximal portion and/or a distal portion in the intramedullary canal by a) inserting one or more screws or posts into fractured bone to limit movement of the proximal portion and/or a distal portion in the intramedullary canal, and/or b) inserting adhesive and/or cement in the intramedullary canal to limit movement of the proximal portion and/or a distal portion in the intramedullary canal.

In another and/or alternative non-limiting object of the present disclosure is the provision of a method for repairing a bone that is fractured further including the steps of a) removing at least a portion of bone marrow from the intramedullary canal prior to insertion of the expandable medical device in the intramedullary canal, and b) at least partially inserting at least a portion of the removed bone marrow into the intramedullary canal after the step of expanding the expandable medical device in the intramedullary canal.

In another and/or alternative non-limiting object of the present disclosure is the provision of a method for repairing a bone that is fractured further including the step of using a sheath to facilitate insertion of the expandable medical device into the intramedullary canal; the sheath includes a tubular structure that has a longitudinal cavity; the longitudinal cavity has a size and shape that is configured to enable the expandable medical device in the unexpanded state to move through the longitudinal cavity; at least a portion of the sheath is optionally formed of an clastic material.

In another and/or alternative non-limiting object of the present disclosure is the provision of a method for repairing a bone that is fractured further including the step of using a guidewire to facilitate insertion of a portion of the expandable medical device into the intramedullary canal; the guidewire has sufficient flexibility and stiffness to enable the expandable medical device in the unexpanded state to move through the intramedullary canal and along the guidewire.

In another and/or alternative non-limiting object of the present disclosure is the provision of a method for repairing a bone that is fractured further including the steps of: a) providing a second expandable medical device; b) inserting a second expandable medical device in an interior of the expanded expandable medical device; and c) expanding the second expandable medical device in an interior of the expanded expandable medical device to increase a strength and/or rigidity about a region of the bone fracture.

In another and/or alternative non-limiting object of the present disclosure is the provision of a method for repairing a bone that is fractured wherein a longitudinal length of the second expandable medical device in an expanded state is less than a longitudinal length of the expanded expandable medical device.

In another and/or alternative non-limiting object of the present disclosure is the provision of a method for repairing a bone that is fractured wherein the second expandable medical device is configured to a) foreshorten when expanded such that the longitudinal length of the expanded second expandable medical device is at least 10% less than a longitudinal length of the second expandable medical device in an unexpanded state.

In another and/or alternative non-limiting object of the present disclosure is the provision of a method for repairing a bone that is fractured wherein ends of the second expandable medical device when the second expandable medical device is expanded inside the expandable medical device do not extend beyond ends of the expandable medical device in the expanded state.

In another and/or alternative non-limiting object of the present disclosure is the provision of an expandable device for treating a fracture site in a fractured bone having an intramedullary canal; the expandable device includes an expandable frame; the expandable frame has an unexpanded shape and size that enables the expandable frame to be inserted into the intramedullary canal; the expandable frame has an expanded shape and size that enables the expandable frame to be secured in the intramedullary canal while traversing a fracture site of the fractured bone; the expandable frame has a longitudinal length that is sufficient to fully span the fracture site; the expandable frame is expandable from a first cross-sectional size in the unexpanded state to a second cross-sectional size in the expanded state; a cross-sectional area of the expandable frame in the second cross-sectional size is larger than a cross-sectional area of the expandable frame in the first cross-sectional size; the longitudinal length of the expandable frame in the unexpanded state is greater than the longitudinal length of the expandable frame in the expanded state; the expandable frame has a side wall that includes a one or more openings; the expandable frame is at least partially formed of metal alloy that includes at least 5 awt. % (e.g., 5-99 awt. % and all values and ranges therebetween) rhenium and additive material; the additive material includes one or more metals that are selected from the group consisting of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and zirconium; the rhenium and the additive material constitutes at least 90 wt. % of the rhenium alloy, and wherein the rhenium alloy optionally includes 0-2 wt. % of a combination of other metals, carbon, oxygen, phosphorous, sulfur, hydrogen and nitrogen; the other metals are metals other than the rhenium and the additive material, and wherein the expandable frame is optionally at least partially coated with a biocompatible material; the biocompatible material optionally includes a) biological agent, b) titanium nitride oxide (TiNOx) coating, c) titanium nitride (TiN) coating, d) chromium nitride (CrN) coating, c) diamond-like carbon (DLC) coating, f) zirconium nitride (ZrN) coating, g) zirconium oxide (ZrO₂) coating, h) zirconium-nitrogen-carbon (ZrNC) coating, i) zirconium OxyCarbide (ZrOC) coating, and/or j) zirconium oxynitride (ZrNxOy) coating, and wherein the expandable frame is optionally at least partially coated with a biocompatible material, and wherein the expandable frame optionally has a generally hollow tubular shape, and wherein said biocompatible material optionally includes no more than 0.1 wt. % nickel and/or no more than 0.1 wt. % cobalt, and wherein said metal alloy of said expandable frame optionally includes no more than 0.1 wt. % nickel and/or no more than 0.1 wt. % cobalt, and wherein said biocompatible material is optionally at least partially coated on a metallic adhesion coating and wherein said metallic adhesion coating optionally includes no more than 0.1 wt. % nickel and/or no more than 0.1 wt. % cobalt.

Other objects, advantages, and novel features of the present disclosure will become apparent from the following detailed description of the disclosure when considered in conjunction with the accompanying drawings.

### BRIEF DESCRIPTION OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for case of recognition in the drawings. Reference may now be made to the drawings, which illustrate various embodiments that the disclosure may take in physical form and in certain parts and arrangement of parts wherein:

FIGS. 1-3 illustrate various non-limiting prior art bone fixation arrangements and non-limiting disadvantages that can be associated with such bone fixation arrangements.

FIG. 4 illustrates a non-limiting expandable medical device in accordance with the present disclosure and a list of non-limiting features of the expandable medical device.

FIGS. 5A-5D illustrate a non-limiting method for using the non-limiting expandable medical device in accordance with the present disclosure to repair a bone fracture.

FIG. 6A-6B illustrate the non-limiting expandable medical device in accordance with the present disclosure in an unexpanded and expanded state and also illustrated that the non-limiting expandable medical device in the expanded state has a shorter longitudinal length than the non-limiting expandable medical device in the unexpanded state.

FIG. 7A-7D illustrate a non-limiting method for using the two non-limiting expandable medical devices in accordance with the present disclosure to repair a bone fracture.

FIG. 8 illustrates non-limiting bones that can be repaired by the use of the expandable medical device.

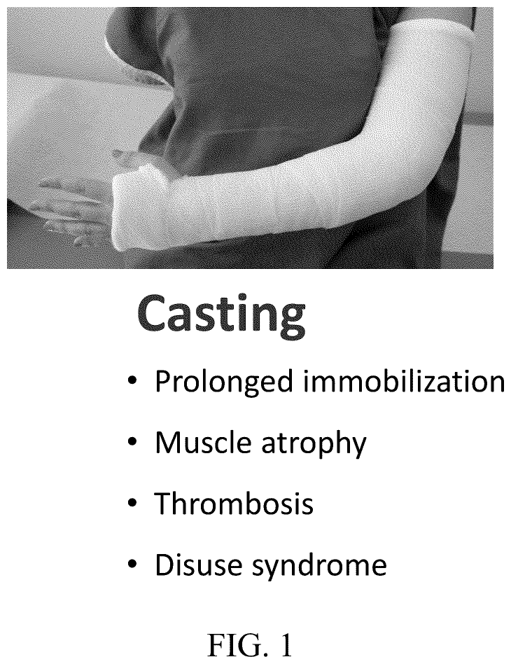

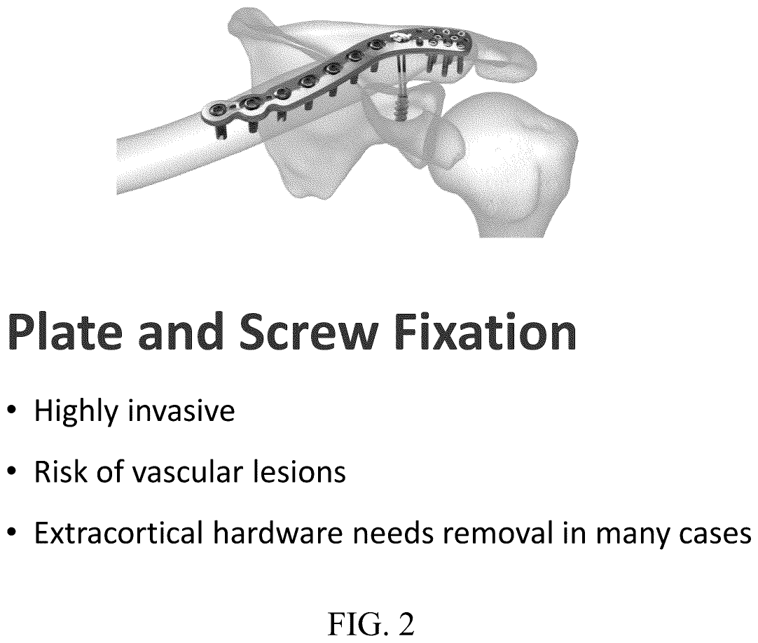

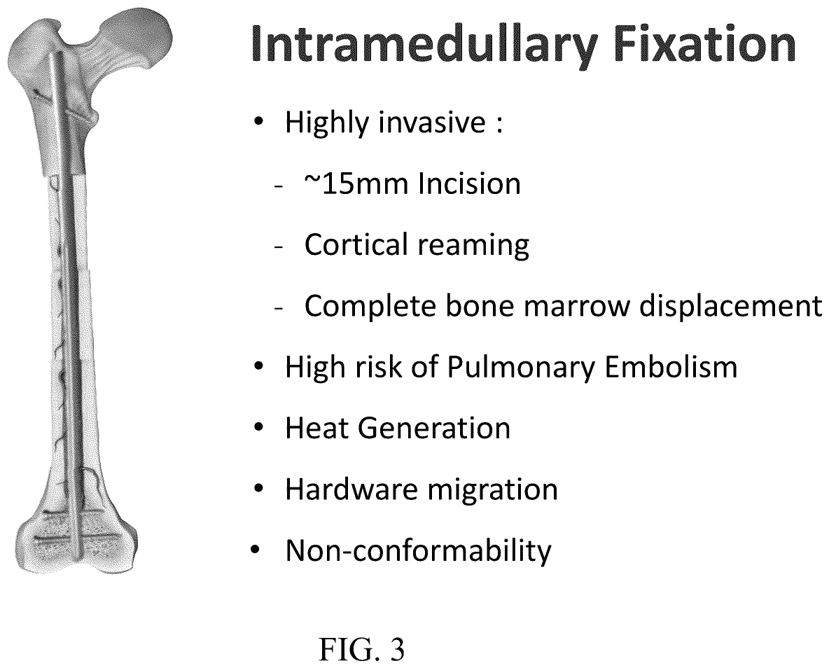

### DETAILED DESCRIPTION OF NON-LIMITING EMBODIMENTS

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

The present disclosure is directed to an expandable medical device that can be partially or fully inserted into a fractured bone to facilitate in the fixation, repair and stabilization of the fractured bone. The expandable medical device is partially or fully formed of a rhenium containing metal alloy, and the rhenium content of the metal alloy can be such that the metal alloy has improved ductility and tensile strength as compared to the same or similar metal alloy that is absent rhenium. The outer surface of the expandable medical device can optionally be coated with an enhancement coating.

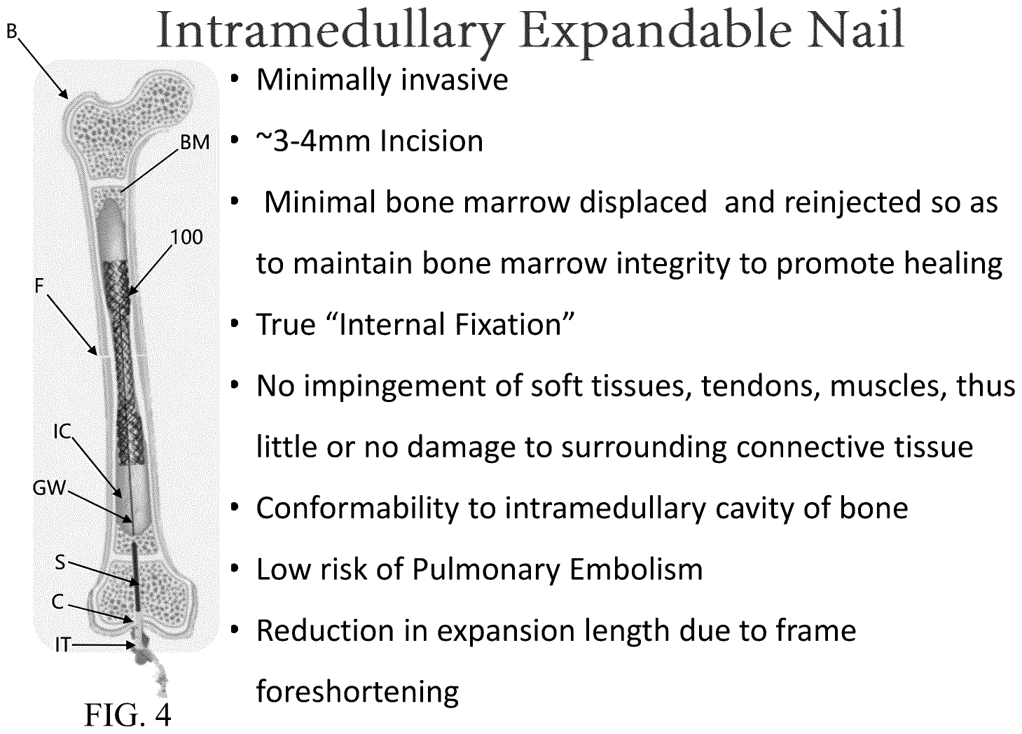

Referring now to FIG. 4, there is illustrated a fractured bone B that includes the expandable medical device 100 positioned in the intramedullary canal IC of the fractured bone B. The expandable medical device 100 is positioned in the intramedullary canal IC such that the expandable medical device 100 overlies the fracture zone F of the fractured bone B. The bone marrow BM has been partially or fully removed in the intramedullary canal IC prior to the insertion of the expandable medical device 100 into the intramedullary canal IC. A guidewire GW is illustrated that can optionally be used to facilitate in guiding the expandable medical device 100 during insertion into the intramedullary canal IC. The base of the fractured bone includes a channel C that has been cut into the fracture bone so that the expandable medical device 100 can be inserted into the intramedullary canal IC. An insertion tool IC is used to inert the expandable medical device 100 into the intramedullary canal IC. The insertion tool IC can optionally include a sheath S that is positioned in channel C and can be used to facilitate in the insertion of the expandable medical device 100 though channel C and into the intramedullary canal IC.

FIG. 4 lists several non-limiting advantages of the expandable medical device 100, namely:
The use of the expandable medical device 100 is minimally invasive. Generally, only a 3-4 mm incision is required in the bone to insert the expandable medical device 100 into the intramedullary canal IC of the fractured bone B. Prior art intramedullary nail generally required an incision into the bone of about 15 mm. The significantly smaller incision in the fractured bone results in improved healing rates of the fractured bone, reduced incidence of damage to the bone during the creation of the incision,
The minimizes the loss of bone marrow during the fracture repair procedure since removed or displaced bone marrow is reinjected into the intramedullary canal after the expandable medical device has been inserted into the intramedullary canal so as to maintain bone marrow integrity and to promote healing of the fractured bone.
The use of the expandable medical device 100 provides for true “Internal Fixation” of the fracture without the need for external devices (e.g., screws, exterior support structures, etc.). The “Internal Fixation” feature of the expandable medical device also results in no impingement of soft tissues, tendons, muscles about the fractured bone, thus little or no damage to surrounding connective tissue about the fracture bone during the repair of the fractured bone.
The use of the expandable medical device 100 provides improved conformity to the shape of the intramedullary canal of the fractured bone as compared to a rigid rod. It is common that the shape of the intramedullary canal of the fractured bone is not linear, especially when the bone is fractured. The flexibility of the frame of the expandable medical device allow the frame to bend during the insertion of the expandable medical device into the intramedullary canal. As such, the flexible frame of the expandable medical device is able to better conform to a non-linear shaped intramedullary canal of the fractured bone, thus resulting in a) improved fracture repair, and b) reduced damage to the intramedullary canal of the fractured bone during insertion of the expandable medical device into the intramedullary canal.
The use of the expandable medical device 100 to repair a fractured bone can result in a lower risk of Pulmonary Embolism.
The expandable medical device 100 is configured to reduce in longitudinal length when the expandable medical device is expanded due to frame foreshortening. Such reduction in longitudinal length of the expandable medical device results in drawing together of the fractured portions of the fractured bone, thereby improving fracture repair.

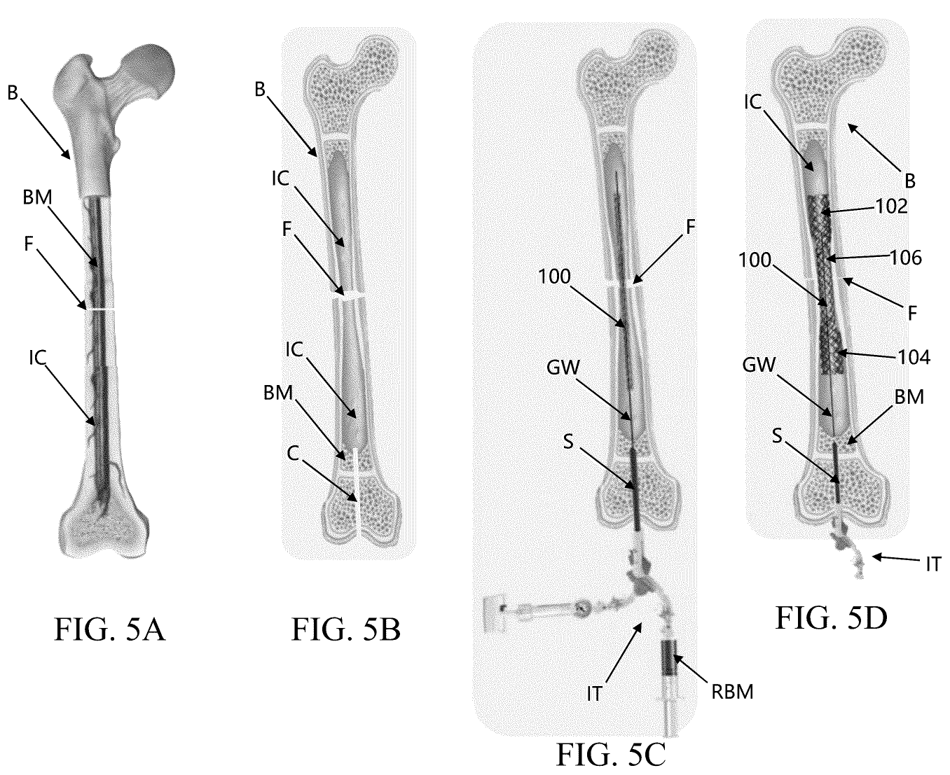

Referring now to FIGS. 5A-5D, there is illustrated a non-limiting method for repairing a fractured bone B by use of the expandable medical device 100. FIG. 5A illustrates a fractured bone B that includes a fracture zone F. The fractured bone F includes an intramedullary canal IC that includes bone marrow BM and other tissue and/or blood vessels. Referring now to FIG. 5A, a channel C in one end of the fractured bone B has been formed. The diameter of the channel can be as small as 2 mm and is typically 3-6 mm (and all values and ranges therebetween). After the channel C is formed, a portion of all of the bone marrow BM and other tissue and/or blood vessels in the intramedullary canal IC can be temporarily removed as illustrated in FIG. 5B. The process to remove the bone marrow BM and other tissue and/or blood vessels in the intramedullary canal IC is known in the art and will not be described herein.

Referring now to FIG. 5C, once the bone marrow BM and other tissue and/or blood vessels in the intramedullary canal IC has been sufficiently removed, an insertion tool IT is used to facilitate in the insertion of the expandable medical device 100 into the intramedullary canal IC. The insertion tool IT can optionally include the use of a sheath S and/or a guidewire GW to facilitate in the insertion of the expandable medical device 100 into the intramedullary canal IC. The sheath is illustrated as being inserted into channel C. The sheath S can optionally be formed of an expandable material that can expand as the expandable medical device 100 is moved through the internal channel of the sheath and into the intramedullary canal IC. The sheath S can be formed of a flexible polymer material. The sheath S can optionally include a shape memory material that cause the sheath to return to its original shape or near original shape as or after the expandable medical device 100 has passed through the internal channel of the sheath. The sheath S is configured to inhibit or prevent damage to the bone marrow that is located about channel C as the expandable medical device 100, while in the crimped or unexpanded state, is passed through the channel C. The sheath S is also configured to inhibit or prevent damage to the expandable medical device 100 as it is passed through the channel C (e.g., front end of expandable medical device gets caught on the bone marrow about the channel, thus bending or otherwise damaging the expandable medical device, etc.). Generally, no more than 50% (e.g., 0-50% and all values and ranges therebetween) of the longitudinal length of the sheath S is inserted into the portion of the intramedullary canal IC where a portion of all of the bone marrow BM and other tissue and/or blood vessels in the intramedullary canal IC has been removed. As illustrated in FIGS. 5C and 5D, the proximal end of sheath S is spaced from the portion of the intramedullary canal IC where a portion of all of the bone marrow BM and other tissue and/or blood vessels in the intramedullary canal IC has been removed; however, this is not required.

The insertion tool IT can optionally include the use of a guidewire GW to facilitate in the insertion of the expandable medical device 100 into the intramedullary canal IC. The guidewire GW is illustrated as being inserted through the channel C and into the portion of the intramedullary canal IC where a portion of all of the bone marrow BM and other tissue and/or blood vessels in the intramedullary canal IC has been removed. If a sheath S is used, the guidewire GW is insert through the internal channel of the sheath S. As illustrated in FIG. 5S, the guidewire GW extends into at least 50% (e.g., 50-100% and all values and ranges therebetween) of the longitudinal length of the portion of the intramedullary canal IC where a portion of all of the bone marrow BM and other tissue and/or blood vessels in the intramedullary canal IC has been removed.

After the optional sheath S and guidewire GW are positioned in the fractured bone B, the expandable medical device 100 is inserted, while in its crimped or unexpanded state, into the portion of the intramedullary canal IC where a portion of all of the bone marrow BM and other tissue and/or blood vessels in the intramedullary canal IC has been removed. When a sheath S is used, the expandable medical device 100 is inserted fully through the internal channel of the sheath S. When a guidewire GW is used, the expandable medical device 100 is inserted about the guidewire GW and then guided along the guidewire and into the intramedullary canal IC until the expandable medical device 100 is properly positioned in the intramedullary canal IC. Generally, the intramedullary canal IC is not inserted past the end of the guidewire GW prior to the expansion of the expandable medical device 100 in the intramedullary canal IC.

Once the expandable medical device 100 is positioned in the intramedullary canal IC, at least a portion of the frame or body of the expandable medical device 100 is expanded form a crimped state to an expanded state in the intramedullary canal IC. As illustrated in FIG. 5D, the posterior portion 102 and anterior portion 104 of the expandable medical device 100 are expanded in the intramedullary canal IC. Such expansion of the posterior and anterior portions 102, 104 of the expandable medical device 100 in the intramedullary canal IC result in the anchoring of the posterior portion 102 of the expandable medical device 100 in one section of the fractured bone B and anchoring of the anterior portion 104 of the expandable medical device 100 in the other section of the fractured bone B. Once both the posterior and anterior portions 102, 104 of the expandable medical device 100 are properly anchored in the intramedullary canal IC, the expandable medical device 100 can optionally be further expanded in the expandable medical device 100 in the intramedullary canal IC. The further expansion of the expandable medical device 100 can occur at the posterior and anterior portions 102, 104 of the expandable medical device 100, and/or in the mid-portion 106 of the expandable medical device 100. The expansion of the posterior and anterior portions 102, 104 of the expandable medical device 100 can be simultaneous or sequential (e.g., partial or full expansion of posterior portion prior to partial or full expansion of anterior portion, partial or full expansion of anterior portion prior to partial or full expansion of posterior portion, etc.). The mid-portion 106 can be optionally expanded after the full expansion of the posterior and anterior portions 102, 104 of the expandable medical device 100

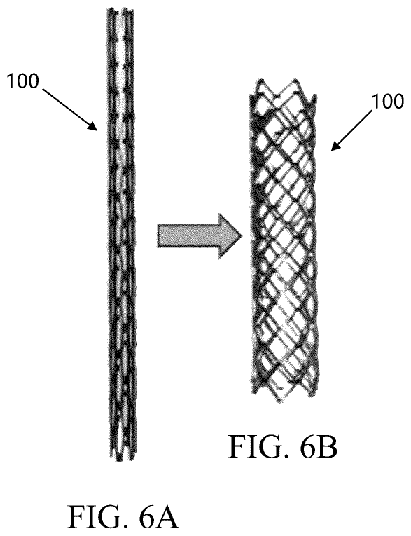

The frame or body of the expandable medical device 100 can be configured to result in foreshortening of the longitudinal length of the expandable medical device when the expandable medical device is expanded from the crimped to the expanded state. Referring now to FIGS. 6A and 6B, there is illustrated a non-limiting configuration of the frame or body of the expandable medical device 100 that is configured to foreshorten when expanded. The frame or body has an open cell configuration and is formed of a plurality of interconnecting struts and/or posts. The interconnecting struts and/or posts can be configured to form a variety of patterns (e.g., zig-zag pattern, saw-tooth pattern, triangular pattern, polygonal pattern, oval pattern, etc.). One or more of the interconnecting struts and/or posts can have the same or different thicknesses and/or cross-sectional shape and/or cross-sectional area. FIG. 6A illustrates the expandable medical device 100 in the crimped or expanded state. FIG. 6B illustrates the expandable medical device 100 in the expanded state. The longitudinal length of the expandable medical device 100 in the expanded state is illustrates as being less than the longitudinal length expandable medical device 100 in the crimped or unexpanded state. In one non-limiting embodiment, the longitudinal length of the expandable medical device 100 in the expanded state is at least 5% (e.g., 5-60% and all values and ranges therebetween) less than the longitudinal length expandable medical device 100 in the crimped or unexpanded state. An inflation device (e.g., expandable balloon, etc.) can be used to expand the expandable medical device 100.

Referring now to FIGS. 5C and 5D, when the expandable medical device 100 is expanded and the longitudinal length of the expandable medical device 100 is reduced, the reduction in longitudinal length of the expandable medical device 100 causes the fractured portions of the fractured bone B to be drawn together thereby facilitating in the repair of the fractured bone B.

The frame or body of the expandable medical device 100 is generally partially or fully formed of a) a refractory metal alloy and/or b) a metal alloy that includes at least 5 atomic weight percent (awt. %) or atomic percent (awt %) rhenium (e.g., 5-99 awt. % rhenium and all values and ranges therebetween). The frame or body of the expandable medical device 100 can be partially or fully formed of a metal alloy that is absent chromium and/or nickel. In one non-limiting embodiment, frame or body of the expandable medical device 100 can be partially or fully formed of a metal alloy that includes at least 5 awt. % (e.g., 5-99 awt. % and all values and ranges therebetween) rhenium, and 0.1-96 wt. % of one or more additives selected from the group of aluminum, boron, beryllium, bismuth, cadmium, calcium, cerium, chromium, cobalt, copper, gallium, gold, hafnium, iridium, iron, lanthanum, lanthanum oxide, lead, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and the metal alloy optionally includes 0-2 wt. % of a combination of other metals (e.g., metals other than additives), carbon, oxygen, phosphorous, sulfur, hydrogen and/or nitrogen.

The frame or body of the expandable medical device 100 can be optionally fully or partially coated with an enhancement coating. Non-limiting enhancement coatings that can be applied to a portion or all of the expandable medical device includes chromium nitride (CrN), diamond-like carbon (DLC), titanium nitride (TiN), titanium nitride oxide (TiNOx), zirconium nitride (ZrN), zirconium oxide (ZrO₂), zirconium-nitrogen-carbon (ZrNC), zirconium OxyCarbide (ZrOC), zirconium oxynitride (ZnNxOy) [e.g., cubic ZrN:O, cubic ZrO₂:N, tetragonal ZrO₂:N, and monoclinic ZrO₂:N phase coatings], and combinations of such coatings. In another non-limiting embodiment, the thickness of the enhancement coating is greater than 1 nanometer (e.g., 2 nanometers to 100 microns and all values and ranges therebetween). In one non-limiting configuration, the 50-100% (and all values ad ranges therebetween) of the outer surface of the frame or body of the expandable medical device 100 is coated with titanium nitride oxide (TiNOx) and/or zirconium oxynitride (ZnNxOy).

Referring again to FIGS. 5C and 5D, after the expandable medical device 100 is expanded in the intramedullary canal IC, a portion of all of the removed bone marrow (RBM) that was removed from the intramedullary canal IC can optionally be reinserted into the intramedullary canal IC via an insertion device (e.g., syringe, etc.). Also, once the expandable medical device 100 is properly positioned and expanded in the intramedullary canal IC of the fractured bone B, a cement, adhesive or other surgical fluid can optionally be used to provide fixation and stabilization of the expandable medical device 100 in the intramedullary canal IC. The cement, adhesive or other surgical fluid, when used, can be inserted in the intramedullary canal IC prior to, during or after the optional insertion of the removed bone marrow (RBM) into the intramedullary canal IC. Generally, the guidewire (GW), when used is partially or fully removed from the intramedullary canal IC prior to the optional insertion of the cement, adhesive or other surgical fluid and/or the removed bone marrow (RBM) into the intramedullary canal IC. The sheath S is removed from the intramedullary canal IC prior to, during or after the full expansion of the expandable medical device 100 in the intramedullary canal IC, and/or the optional insertion of the cement, adhesive or other surgical fluid and/or the removed bone marrow (RBM) into the intramedullary canal IC. After the insertion tool IT is removed from channel C, channel C can optionally be sealed by a scaling material (e.g., cement, adhesive or other surgical fluid, bone fragments, etc.) after the full expansion of the expandable medical device 100 in the intramedullary canal IC, and the optional insertion of the cement, adhesive or other surgical fluid and/or the removed bone marrow (RBM) into the intramedullary canal IC.

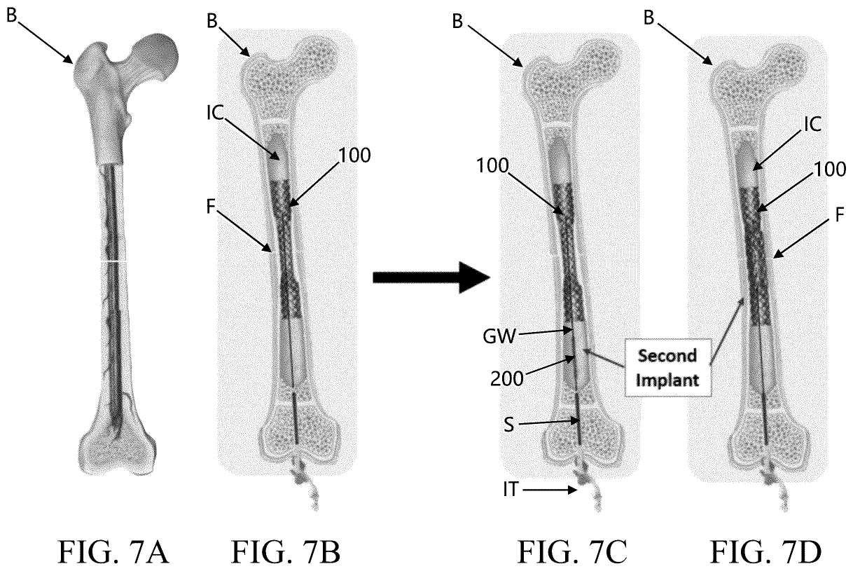

Referring now to FIGS. 7A-7D, there is illustrated a modified procedure to the bone repair procedure illustrated in FIGS. 5A-5D. The modified procedure includes the procedure discussed above with regard to FIGS. 5A-5D, but includes the use of a second expandable medical device 200. A second expandable medical device 200 can be optionally inserted in the interior of a first expanded expandable medical device 100 to increase the strength and/or rigidity of the expandable medical device system at the fracture F of the fractured bone B. For example, the first expandable medical device 100 can be inserted and expanded in the intramedullary canal IC as discussed above with respect to FIGS. 5A-5D. Thereafter, a second expandable medical device 200 can be inserted into the interior of the expanded first expandable medical device 200. Generally, the longitudinal length of the expanded second expandable medical device 200 is less (e.g., 20-80% less and all values and ranges therebetween) than the longitudinal length of the expanded first expandable medical device 200.

Generally, the longitudinal length of the unexpanded second expandable medical device 200 is equal to or less (e.g., 20-80% less and all values and ranges therebetween) than the longitudinal length of the unexpanded first expandable medical device 200. In one non-limiting configuration, the longitudinal length of the unexpanded second expandable medical device 200 is less than the longitudinal length of the unexpanded first expandable medical device 200 such that when the unexpanded second expandable medical device 200 is inserted inside the expanded first expandable medical device 100, the ends of the unexpanded second expandable medical device 200 are spaced inwardly from the ends of the expanded first expandable medical device 100. In another non-limiting configuration, when the first and second expandable medical devices 100, 200 are expanded, the ends of the expanded second expandable medical device 200 are spaced inwardly from the ends of the expanded first expandable medical device 100.

As illustrated in FIG. 7D, the expanded second expandable medical device 200 traverses the length of the bone fracture. Generally, 10-70% of the longitudinal length (and all values and ranges therebetween) of one side of the expanded second expandable medical device 200 is located on one side of the fracture F, and 10-70% of the longitudinal length (and all values and ranges therebetween) of the other side of the expanded second expandable medical device 200 is located on the other side of the fracture F. As illustrated in FIG. 7C, the second expandable medical device 200 is inserted into the interior of the first expanded second expandable medical device 100 prior to the expansion of the second expandable medical device 200. The second expandable medical device 200 can be configured to a) foreshorten when expanded such that the longitudinal length of the expanded second expandable medical device is at least 10% less (e.g., 10-60% less and all values and ranges therebetween) than a longitudinal length of the crimped or non-expanded second expandable medical device 200, or b) not or substantially not foreshorten when expanded such that the longitudinal length of the expanded second expandable medical device is no more than 10% less (e.g., 0-10% less and all values and ranges therebetween) than a longitudinal length of the crimped or non-expanded second expandable medical device 200. When the expanded second expandable medical device 200 is configured to foreshorten, the reduced longitudinal length of the expanded second expandable medical device 200 can facilitate in further drawing the fractured portion of the fractured bone B together. The use of the expanded second expandable medical device 200 results in increased rigidity and/or strength in the region of the two expanded expandable medical devices 100, 200 so as to provide additional supported to the fracture region of the fractured bone B. The material used to form the first and second expandable medical devices 100, 200 can be the same or different. One or both of the first and second expandable medical devices 100, 200 can include an enhancement coating and/or biological agent.

After the second expandable medical device 200 has been expanded, a portion of all of the removed bone marrow (RBM) that was removed from the intramedullary canal IC can optionally be reinserted into the intramedullary canal IC via an insertion device (e.g., syringe, etc.). Also, once the expandable medical devices 100, 200 are properly positioned and expanded in the intramedullary canal IC of the fractured bone B, a cement, adhesive or other surgical fluid can optionally be used to provide fixation and stabilization of the expandable medical devices 100, 200 in the intramedullary canal IC. The cement, adhesive or other surgical fluid, when used, can be inserted in the intramedullary canal IC prior to, during or after the optional insertion of the removed bone marrow (RBM) into the intramedullary canal IC. Generally, the guidewire (GW), when used is partially or fully removed from the intramedullary canal IC prior to the optional insertion of the cement, adhesive or other surgical fluid and/or the removed bone marrow (RBM) into the intramedullary canal IC. The sheath S is removed from the intramedullary canal IC prior to, during or after the full expansion of the expandable medical devices 100, 200 in the intramedullary canal IC, and/or the optional insertion of the cement, adhesive or other surgical fluid and/or the removed bone marrow (RBM) into the intramedullary canal IC. After the insertion tool IT is removed from channel C, channel C can optionally be sealed by a scaling material (e.g., cement, adhesive or other surgical fluid, bone fragments, etc.) after the full expansion of the expandable medical devices 100, 200 in the intramedullary canal IC, and the optional insertion of the cement, adhesive or other surgical fluid and/or the removed bone marrow (RBM) into the intramedullary canal IC.

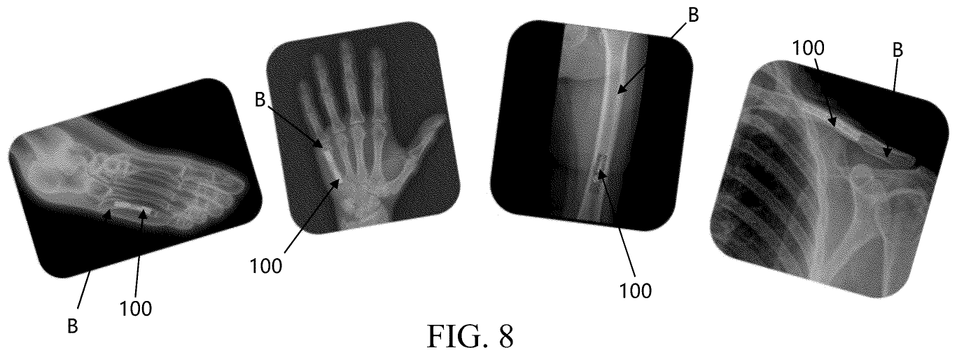

Referring now to FIG. 8, there is illustrated several different non-limiting fractured bones B than can be repaired with one or two expandable medical devices in accordance with the present disclosure.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall therebetween.

To aid the Patent Office and any readers of this application and any resulting patent in interpreting the claims appended hereto, applicants do not intend any of the appended claims or claim elements to invoke 35 U.S.C. 112(f) unless the words “means for” or “step for” are explicitly used in the particular claim.

1. An expandable device for treating a fracture site in a fractured bone having an intramedullary canal; said expandable device includes an expandable frame that is configured to be inserted into the intramedullary canal of a fractured bone; said expandable frame has an unexpanded shape and size that enables said expandable frame to be inserted into the intramedullary canal; said expandable frame has an expanded shape and size that enables said expandable frame to be secured in the intramedullary canal while traversing a fracture site of the fractured bone; said expandable frame has a longitudinal length that is sufficient to fully span the fracture site; said expandable frame is expandable from a first cross-sectional size in said unexpanded state to a second cross-sectional size in said expanded state; a cross-sectional area of said expandable frame in said second cross-sectional size is larger than a cross-sectional area of said expandable frame in said first cross-sectional size; said longitudinal length of said expandable frame in said unexpanded state is greater than said longitudinal length of said expandable frame in said expanded state; said expandable frame has a side wall that includes a plurality of openings; at least 50 wt. % of said expandable frame is formed of metal alloy that includes at least 5 awt. % rhenium and additive material; said additive material includes one or more metals that are selected from the group consisting of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and zirconium; said rhenium and said additive material constitutes at least 90 wt. % of said metal alloy.

2. The expandable device as defined in claim 1, wherein said rhenium alloy includes 0-2 wt. % of a combination of other metals, carbon, oxygen, phosphorous, sulfur, hydrogen and nitrogen; said other metals are metals other than said rhenium and said additive material.

3. The expandable device as defined in claim 1, wherein said expandable frame is at least partially coated with a biocompatible material; said biocompatible material includes a) biological agent, b) titanium nitride oxide (TiNOx) coating, c) titanium nitride (TiN) coating, d) chromium nitride (CrN) coating, e) diamond-like carbon (DLC) coating, f) zirconium nitride (ZrN) coating, g) zirconium oxide (ZrO2) coating, h) zirconium-nitrogen-carbon (ZrNC) coating, i) zirconium OxyCarbide (ZrOC) coating, and/or j) zirconium oxynitride (ZrNxOy) coating.

4. The expandable device as defined in claim 2, wherein said expandable frame is at least partially coated with a biocompatible material; said biocompatible material includes a) biological agent, b) titanium nitride oxide (TiNOx) coating, c) titanium nitride (TiN) coating, d) chromium nitride (CrN) coating, e) diamond-like carbon (DLC) coating, f) zirconium nitride (ZrN) coating, g) zirconium oxide (ZrO2) coating, h) zirconium-nitrogen-carbon (ZrNC) coating, i) zirconium OxyCarbide (ZrOC) coating, and/or j) zirconium oxynitride (ZrNxOy) coating.

5. The expandable device as defined in claim 1, wherein said expandable frame is at least partially coated with a biocompatible material; said biocompatible material includes a) biological agent, b) titanium nitride oxide (TiNOx) coating, and/or c) zirconium oxynitride (ZrNxOy) coating.

6. The expandable device as defined in claim 2, wherein said expandable frame is at least partially coated with a biocompatible material; said biocompatible material includes a) biological agent, b) titanium nitride oxide (TiNOx) coating, and/or c) zirconium oxynitride (ZrNxOy) coating.

7. The expandable device as defined in claim 5, wherein said biocompatible material includes a) TiNOx coating and/or b) zirconium oxynitride (ZrNxOy) coating.

8. The expandable device as defined in claim 6, wherein said biocompatible material includes a) TiNOx coating and/or b) zirconium oxynitride (ZrNxOy) coating.

9. The expandable device as defined in claim 1, wherein said expandable frame has a generally hollow tubular shape.

10. The expandable device as defined in claim 2, wherein said expandable frame has a generally hollow tubular shape.

11. A method for repairing a bone that is fractured comprising:
providing a fractured bone that includes first and second bone portions and a fracture site that is located between said first and second bone portions; said fracture site has a fracture site width; each of said first and second bone portions of said fractured bone includes an intramedullary canal;
providing an expandable device; said expandable device includes an expandable frame; said expandable frame has an unexpanded shape and size that enables said expandable frame to be inserted into the intramedullary canal; said expandable frame has an expanded shape and size that enables said expandable frame to be secured in the intramedullary canal while traversing a fracture site of the fractured bone; said expandable frame has a longitudinal length that is sufficient to fully span said fracture site; said expandable frame is expandable from a first cross-sectional size in said unexpanded state to a second cross-sectional size in said expanded state; a cross-sectional area of said expandable frame in said second cross-sectional size is larger than a cross-sectional area of said expandable frame in said first cross-sectional size; said longitudinal length of said expandable frame in said unexpanded state is greater than said longitudinal length of said expandable frame in said expanded state; said expandable frame has a side wall that includes a one or more openings; said expandable frame is at least partially formed of metal alloy that includes at least 5 awt. % rhenium and additive material; said additive material includes one or more metals that are selected from the group consisting of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and zirconium; said rhenium and said additive material constitutes at least 90 wt. % of said rhenium alloy;
inserting said expandable device in said intramedullary canal while said expandable device is in said unexpanded state such that at least a portion of said expandable device is positioned in said first and second bone portions and traverses said fracture site; and
expanding said expandable device in said intramedullary canal to said expanded state to cause said longitudinal length of said first expandable device to shorten and to cause said first expandable device to at least partially repair said fractured bone; and
wherein expansion of said expandable device causes said first and second bone portions to thereby cause a reduction in said fracture site width.

12. The method as defined in claim 11, wherein said expandable device includes a proximal portion, a distal portion and a mid-portion; and wherein said step of expanding includes expanding said proximal portion and/or a distal portion of said expandable device prior to expanding said mid-portion; and wherein prior expansion of said proximal portion and/or a distal portion causes said proximal portion and/or a distal portion to be at least partially anchored in said intramedullary canal prior to expansion of said mid-portion.

13. The method as defined in claim 11, wherein said expandable device includes a proximal portion, a distal portion and a mid-portion; and further including the step of securing said proximal portion and/or a distal portion in said intramedullary canal by a) inserting one or more screws or posts into fractured bone to limit movement of said proximal portion and/or a distal portion in said intramedullary canal, and/or b) inserting adhesive and/or cement in said intramedullary canal to limit movement of said proximal portion and/or a distal portion in said intramedullary canal.

14. The method as defined in claim 12, wherein said expandable device includes a proximal portion, a distal portion and a mid-portion; and further including the step of securing said proximal portion and/or a distal portion in said intramedullary canal by a) inserting one or more screws or posts into fractured bone to limit movement of said proximal portion and/or a distal portion in said intramedullary canal, and/or b) inserting adhesive and/or cement in said intramedullary canal to limit movement of said proximal portion and/or a distal portion in said intramedullary canal.

15. The method as defined in claim 11, further including the steps of a) removing at least a portion of bone marrow from said intramedullary canal prior to insertion of said expandable device in said intramedullary canal, and b) at least partially inserting at least a portion of said removed bone marrow into said intramedullary canal after said step of expanding said expandable device in said intramedullary canal.

16. The method as defined in claim 12, further including the steps of a) removing at least a portion of bone marrow from said intramedullary canal prior to insertion of said expandable device in said intramedullary canal, and b) at least partially inserting at least a portion of said removed bone marrow into said intramedullary canal after said step of expanding said expandable device in said intramedullary canal.

17. The method as defined in claim 11, wherein said rhenium alloy includes 0-2 wt. % of a combination of other metals, carbon, oxygen, phosphorous, sulfur, hydrogen and nitrogen; said other metals are metals other than said rhenium and said additive material.

18. The method as defined in claim 12, wherein said rhenium alloy includes 0-2 wt. % of a combination of other metals, carbon, oxygen, phosphorous, sulfur, hydrogen and nitrogen; said other metals are metals other than said rhenium and said additive material.

19. The method as defined in claim 11, wherein said expandable frame is at least partially coated with a biocompatible material; said biocompatible material includes a) biological agent, b) titanium nitride oxide (TiNOx) coating, c) titanium nitride (TiN) coating, d) chromium nitride (CrN) coating, e) diamond-like carbon (DLC) coating, f) zirconium nitride (ZrN) coating, g) zirconium oxide (ZrO₂) coating, h) zirconium-nitrogen-carbon (ZrNC) coating, i) zirconium OxyCarbide (ZrOC) coating, and/or j) zirconium oxynitride (ZrNxOy) coating.

20. The method as defined in claim 12, wherein said expandable frame is at least partially coated with a biocompatible material; said biocompatible material includes a) biological agent, b) titanium nitride oxide (TiNOx) coating, c) titanium nitride (TiN) coating, d) chromium nitride (CrN) coating, e) diamond-like carbon (DLC) coating, f) zirconium nitride (ZrN) coating, g) zirconium oxide (ZrO₂) coating, h) zirconium-nitrogen-carbon (ZrNC) coating, i) zirconium OxyCarbide (ZrOC) coating, and/or j) zirconium oxynitride (ZrNxOy) coating.

21. The method as defined in claim 19, wherein said biocompatible material includes a) TiNOx coating and/or b) zirconium oxynitride (ZrNxOy) coating.

22. The method as defined in claim 20, wherein said biocompatible material includes a) TiNOx coating and/or b) zirconium oxynitride (ZrNxOy) coating.

23. The method as defined in claim 11, wherein said expandable frame has a generally hollow tubular shape.

24. The method as defined in claim 12, wherein said expandable frame has a generally hollow tubular shape.

25. The method as defined in claim 11, further including the step of using a sheath to facilitate insertion of said expandable device into said intramedullary canal; said sheath includes a tubular structure that has a longitudinal cavity; said longitudinal cavity has a size and shape that is configured to enable said expandable device in said unexpanded state to move through said longitudinal cavity; at least a portion of said sheath is optionally formed of an elastic material.

26. The method as defined in claim 12, further including the step of using a sheath to facilitate insertion of said expandable device into said intramedullary canal; said sheath includes a tubular structure that has a longitudinal cavity; said longitudinal cavity has a size and shape that is configured to enable said expandable device in said unexpanded state to move through said longitudinal cavity; at least a portion of said sheath is optionally formed of an elastic material.

27. The method as defined in claim 11, further including the step of using a guidewire to facilitate insertion of a portion of said expandable device into said intramedullary canal; said guidewire has sufficient flexibility and stiffness to enable said expandable device in said unexpanded state to move through said intramedullary canal and along said guidewire.

28. The method as defined in claim 12, further including the step of using a guidewire to facilitate insertion of a portion of said expandable device into said intramedullary canal; said guidewire has sufficient flexibility and stiffness to enable said expandable device in said unexpanded state to move through said intramedullary canal and along said guidewire.

29. The method as defined in claim 11, further including the steps of:
providing a second expandable device; said second expandable device includes a second expandable frame with at least one opening; said second expandable frame, when oriented in an unexpanded shape and size, enables said second expandable device to be insert into said intramedullary canal; said expandable frame is configured to be expanded to an expanded shape and size; said second expandable frame has a longitudinal length that is sufficient to fully span said fracture site; said second expandable frame is expandable from a first cross-sectional size in said unexpanded state to a second cross-sectional size; a cross-sectional area of said second expandable frame in said second cross-sectional size is larger than a cross-sectional area of said second expandable frame in said first cross-sectional size; said longitudinal length of said second expandable frame in said unexpanded state is greater than said longitudinal length of said second expandable frame in said expanded state; said second expandable frame has a side wall that includes a plurality of openings; said second expandable frame is at least partially formed of metal alloy that includes at least 5 awt. % rhenium and additive material; said additive material includes one or more metals that are selected from the group consisting of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and zirconium; said rhenium and said additive material constitutes at least 90 wt. % of said rhenium alloy;
inserting a second expandable device in an interior of said expanded expandable device; and
expanding said second expandable device in an interior of said expanded expandable device to increase a strength and/or rigidity about a region of said bone fracture.

30. The method as defined in claim 12, further including the steps of:
providing a second expandable device; said second expandable device includes a second expandable frame with an open cell configuration; said second expandable frame includes a plurality of interconnected struts; said second expandable frame, when oriented in an unexpanded shape and size, enables said second expandable device to be insert into said intramedullary canal; said expandable frame is configured to be expanded to an expanded shape and size; said second expandable frame has a longitudinal length that is sufficient to fully span said fracture site; said second expandable frame is expandable from a first cross-sectional size in said unexpanded state to a second cross-sectional size; a cross-sectional area of said second expandable frame in said second cross-sectional size is larger than a cross-sectional area of said second expandable frame in said first cross-sectional size; said longitudinal length of said second expandable frame in said unexpanded state is greater than said longitudinal length of said second expandable frame in said expanded state; said second expandable frame has a side wall that includes a plurality of openings; said second expandable frame is at least partially formed of metal alloy that includes at least 5 awt. % rhenium and additive material; said additive material includes one or more metals that are selected from the group consisting of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and zirconium; said rhenium and said additive material constitutes at least 90 wt. % of said rhenium alloy;
inserting a second expandable device in an interior of said expanded expandable device; and
expanding said second expandable device in an interior of said expanded expandable device to increase a strength and/or rigidity about a region of said bone fracture.

31. The method as defined in claim 29, wherein a longitudinal length of said second expandable device in an expanded state is less than a longitudinal length of the expanded expandable device.

32. The method as defined in claim 30, wherein a longitudinal length of said second expandable device in an expanded state is less than a longitudinal length of the expanded expandable device.

33. The method as defined in claim 29, wherein said second expandable device is configured to a) foreshorten when expanded such that said longitudinal length of said expanded second expandable device is at least 10% less than a longitudinal length of said second expandable device in an unexpanded state.

34. The method as defined in claim 30, wherein said second expandable device is configured to a) foreshorten when expanded such that said longitudinal length of said expanded second expandable device is at least 10% less than a longitudinal length of said second expandable device in an unexpanded state.

35. The method as defined in claim 29, wherein ends of said second expandable device when said second expandable device is expanded inside said expandable device do not extend beyond ends of said expandable device in said expanded state.

36. The method as defined in claim 30, wherein ends of said second expandable device when said second expandable device is expanded inside said expandable device do not extend beyond ends of said expandable device in said expanded state.

37. The method as defined in claim 29, wherein said second expandable frame is at least partially coated with a biocompatible material; said biocompatible material includes a) biological agent, b) titanium nitride oxide (TiNOx) coating, c) titanium nitride (TiN) coating, d) chromium nitride (CrN) coating, e) diamond-like carbon (DLC) coating, f) zirconium nitride (ZrN) coating, g) zirconium oxide (ZrO2) coating, h) zirconium-nitrogen-carbon (ZrNC) coating, i) zirconium OxyCarbide (ZrOC) coating, and/or j) zirconium oxynitride (ZrNxOy) coating.

38. The method as defined in claim 30, wherein said second expandable frame is at least partially coated with a biocompatible material; said biocompatible material includes a) biological agent, b) titanium nitride oxide (TiNOx) coating, c) titanium nitride (TiN) coating, d) chromium nitride (CrN) coating, e) diamond-like carbon (DLC) coating, f) zirconium nitride (ZrN) coating, g) zirconium oxide (ZrO2) coating, h) zirconium-nitrogen-carbon (ZrNC) coating, i) zirconium OxyCarbide (ZrOC) coating, and/or j) zirconium oxynitride (ZrNxOy) coating.

39. An expandable device for treating a fracture site in a fractured bone having an intramedullary canal; said expandable device includes an expandable frame that is configured to be inserted into the intramedullary canal of a fractured bone; said expandable frame has an open cell configuration; said second expandable frame includes a plurality of interconnected struts; said expandable frame has an unexpanded shape and size that enables said expandable frame to be inserted into the intramedullary canal; said expandable frame has an expanded shape and size that enables said expandable frame to be secured in the intramedullary canal while traversing a fracture site of the fractured bone; said expandable frame has a longitudinal length that is sufficient to fully span the fracture site; said expandable frame is expandable from a first cross-sectional size in said unexpanded state to a second cross-sectional size in said expanded state; a cross-sectional area of said expandable frame in said second cross-sectional size is larger than a cross-sectional area of said expandable frame in said first cross-sectional size; said longitudinal length of said expandable frame in said unexpanded state is greater than said longitudinal length of said expandable frame in said expanded state; said expandable frame has a side wall that includes a plurality of open cells; said expandable frame has a generally hollow tubular shape; at least 50 wt. % of said expandable frame is formed of metal alloy that includes a) at least 15 awt. % rhenium and additive material, and wherein said additive material includes one or more metals that are selected from the group consisting of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and zirconium, and wherein said rhenium and said additive material constitutes at least 90 wt. % of said metal alloy; at least a portion of an outer surface of said expandable frame includes a coating material selected from the group consisting of titanium nitride oxide (TiNOx) coating, titanium nitride (TiN) coating, chromium nitride (CrN) coating, zirconium nitride (ZrN) coating, zirconium oxide (ZrO₂) coating, zirconium-nitrogen-carbon (ZrNC) coating, zirconium OxyCarbide (ZrOC) coating, and/or zirconium oxynitride (ZrNxOy) coating.

40. The expandable device as defined in claim 39, wherein said coating material includes titanium nitride oxide (TiNOx) coating and/or zirconium oxynitride (ZrNxOy) coating.

41. A method for repairing a bone that is fractured comprising:
providing a fractured bone that includes first and second bone portions and a fracture site that is located between said first and second bone portions; said fracture site has a fracture site width; each of said first and second bone portions of said fractured bone includes an intramedullary canal;
providing a first expandable device; said first expandable device includes a first expandable frame that is configured to be inserted into the intramedullary canal of a fractured bone; said first expandable frame has an open cell configuration; said first expandable frame includes a plurality of interconnected struts; said first expandable frame has an unexpanded shape and size that enables said first expandable frame to be inserted into the intramedullary canal; said first expandable frame has an expanded shape and size that enables said first expandable frame to be secured in the intramedullary canal while traversing a fracture site of the fractured bone; said first expandable frame has a longitudinal length that is sufficient to fully span the fracture site; said first expandable frame is expandable from a first cross-sectional size in said unexpanded state to a second cross-sectional size in said expanded state; a cross-sectional area of said first expandable frame in said second cross-sectional size is larger than a cross-sectional area of said first expandable frame in said first cross-sectional size; said longitudinal length of said first expandable frame in said unexpanded state is greater than said longitudinal length of said first expandable frame in said expanded state; said first expandable frame has a side wall that includes a plurality of open cells; said first expandable frame has a generally hollow tubular shape; at least 50 wt. % of said first expandable frame is formed of metal alloy that includes a) at least 15 awt. % rhenium and additive material, and wherein said additive material includes one or more metals that are selected from the group consisting of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and zirconium, and wherein said rhenium and said additive material constitutes at least 90 wt. % of said metal alloy; at least a portion of an outer surface of said first expandable frame includes a coating material selected from the group consisting of titanium nitride oxide (TiNOx) coating, titanium nitride (TiN) coating, chromium nitride (CrN) coating, zirconium nitride (ZrN) coating, zirconium oxide (ZrO₂) coating, zirconium-nitrogen-carbon (ZrNC) coating, zirconium OxyCarbide (ZrOC) coating, and/or zirconium oxynitride (ZrNxOy) coating;
inserting said first expandable device in said intramedullary canal while said first expandable device is in said unexpanded state such that at least a portion of said first expandable device is positioned in said first and second bone portions and traverses said fracture site;
expanding said first expandable device in said intramedullary canal to said expanded state and to cause said longitudinal length of said first expandable device to shorten and to cause said first expandable device to at least partially repair said fractured bone;
providing a second expandable device; said second expandable device includes a second expandable frame with an open cell configuration; said second expandable frame includes a plurality of interconnected struts; said second expandable frame, when oriented in an unexpanded shape and size, enables said second expandable device to be insert into said intramedullary canal; said expandable frame is configured to be expanded to an expanded shape and size; said second expandable frame has a longitudinal length that is sufficient to fully span said fracture site; said second expandable frame is expandable from a first cross-sectional size in said unexpanded state to a second cross-sectional size; a cross-sectional area of said second expandable frame in said second cross-sectional size is larger than a cross-sectional area of said second expandable frame in said first cross-sectional size; said longitudinal length of said second expandable frame in said unexpanded state is greater than said longitudinal length of said second expandable frame in said expanded state; said second expandable frame has a side wall that includes a plurality of open cells; said second expandable frame has a generally hollow tubular shape; at least 50 wt. % of said second expandable frame is formed of metal alloy that includes a) at least 15 awt. % rhenium and additive material, and wherein said additive material includes one or more metals that are selected from the group consisting of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and zirconium, and wherein said rhenium and said additive material constitutes at least 90 wt. % of said metal alloy; at least a portion of an outer surface of said second expandable frame includes a coating material selected from the group consisting of titanium nitride oxide (TiNOx) coating, titanium nitride (TiN) coating, chromium nitride (CrN) coating, zirconium nitride (ZrN) coating, zirconium oxide (ZrO₂) coating, zirconium-nitrogen-carbon (ZrNC) coating, zirconium OxyCarbide (ZrOC) coating, and/or zirconium oxynitride (ZrNxOy) coating; said longitudinal length of said second expandable frame in said unexpanded state is less than said longitudinal length of said first expandable frame in both said expanded and unexpanded state;
inserting said second expandable device in said intramedullary canal and into an interior of said expanded first expandable device while said second expandable device is in said unexpanded state such that at least a portion of said second expandable device is positioned in said first and second bone portions and traverses said fracture site and wherein both ends of said second expandable device are spaced inwardly from ends of said expanded first expandable device;
expanding said second expandable device in said intramedullary canal to said expanded state and to cause said longitudinal length of said second expandable device to shorten and to cause said second expandable device to at least partially repair said fractured bone and to increase a strength and/or rigidity about a region of said bone fracture; and
wherein expansion of said first expandable device causes said first and second bone portions to move together and thereby cause a reduction in said fracture site width; and
wherein expansion of said second expandable device in said intramedullary canal and in said interior of said expanded first expandable device results in both of said ends of said second expandable device to be spaced inwardly from said ends of said expanded first expandable device.

42. The method as defined in claim 41, wherein said coating material on said first and second expandable frames includes titanium nitride oxide (TiNOx) coating and/or zirconium oxynitride (ZrNxOy) coating.
